SEQUENCE LISTING (11 sequences)

SEQ ID NO: 1 - protein, 9 aa, Homo sapiens
VMAPRTLFL

SEQ ID NO: 2 - protein, 9 aa, Homo sapiens
VMAPRTLIL

SEQ ID NO: 3 - protein, 9 aa, Homo sapiens
VMAPRTLVL

SEQ ID NO: 4 - protein, 9 aa, Homo sapiens
NLVPMVATV

SEQ ID NO: 5 - protein, 10 aa, Homo sapiens
TPRVTGGGAM

SEQ ID NO: 6 - protein, 24 aa, Homo sapiens
MVVMAPRTLFLLLSGALTLTETWA

SEQ ID NO: 7 - DNA, 22 bp, Artificial Sequence
  Description of Artificial Sequence Synthetic primer
gggaccagacgggagattcctg

SEQ ID NO: 8 - DNA, 18 bp, Artificial Sequence
  Description of Artificial Sequence Synthetic primer
agctcgcatccgtgcatc

SEQ ID NO: 9 - DNA, 20 bp, Artificial Sequence
  Description of Artificial Sequence Synthetic oligonucleotide
cgccatcctcgaagaagacg

SEQ ID NO: 10 - DNA, 20 bp, Artificial Sequence
  Description of Artificial Sequence Synthetic oligonucleotide
gttcatttgacactggccac

SEQ ID NO: 11 - DNA, 20 bp, Artificial Sequence
  Description of Artificial Sequence Synthetic oligonucleotide
acttggatcccgatctccac

(19) United States
(12) Patent Application Publication
(10) Pub. No.: US 2024/0261402 A1
(43) Pub. Date: Aug. 8, 2024

(54) NKG2C+ T CELLS AND METHODS OF USE THEREOF

(71) Applicant: Memorial Sloan Kettering Cancer Center, New York, NY (US)

(72) Inventors: Katharine C Hsu, New York, NY (US); Mohammed Kazim Panjwani, New York, NY (US); Rosa Sottile, New York, NY (US); Jean-Benoit Luduec, New York, NY (US)

(21) Appl. No.: 18/560,436

(22) PCT Filed: May 13, 2022
(86) PCT No.: PCT/US2022/029309

§ 371 Date: Nov. 13, 2023

Related U.S. Application Data
(60) Provisional application No. 63/188,435, filed on May 13, 2021.

(51) Int. Cl. A61K 39/00; C07K 14/725; C07K 16/28; C12N 5/0783

(52) U.S. Cl. CPC A61K 39/4611; A61K 39/4631; A61K 39/4632; A61K 39/464412; C07K 14/7051; C07K 16/2803; C12N 5/0636; C07K 2317/73; C12N 2510/00

(57) ABSTRACT

The present invention provides NKG2C+CD8+ T cells, including engineered NKG2C+CD8+ T cells expressing chimeric antigen receptor (CAR) A molecules or transgenic T cell receptors, compositions comprising such cells, methods of generating such cells from conventional CD8+ T cells, and methods of using such cells, for example in adoptive cell therapy methods.

### CROSS-REFERENCE TO RELATED APPLICATIONS

This application claims the benefit of priority of U.S. Provisional Patent Application No. 63/188,435 filed on May 13, 2021, the content of which is hereby incorporated by reference in its entirety.

### STATEMENT REGARDING FEDERALLY SPONSORED RESEARCH

This invention was made with government support under grant number AI150999 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on May 13, 2022, is named MSKCC_053_WO2_SL.txt and is 2,403 bytes in size.

### INCORPORATION BY REFERENCE

For the purpose of only those jurisdictions that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention.

### BACKGROUND

Adoptive cell therapy (ACT) for cancer treatment involves administering immune cells to patients to attack their tumor cells. Some ACT methods involve administering T cells that have been genetically modified, for example to express chimeric antigen receptors (CARs) or transgenic T cell receptors (TCRs). While there has been some clinical success with such methods, the technology is in its early stages and is not always effective. And in some cases, such methods result undesired side effects. As such, there remains a need in the art for new and improved cell populations for use in ACT methods. The present invention addresses this and other needs in the art.

### SUMMARY OF THE INVENTION

The present invention is based, in part, on a series of important discoveries that are described in more detail in the Examples sections of this patent specification. For example, novel populations of T cells have been identified that are NKG2C+ CD8+, have the capacity to proliferate without upregulation of the exhaustion marker PD1, and that exhibit high effector function against tumor cells and virus infected cells. It has also been discovered that such NKG2C+ CD8+ T cells can be generated from NKG2C−CD8+ T cells by knocking out expression of BCL11B. Furthermore, it has also been discovered that these NKG2C+ CD8+ T cells can be successfully engineered to express an anti-CD19 chimeric antigen receptor (CAR) and that the resulting engineered CAR T cells display superior tumor killing activity as compared to NKG2C−CD8+ CAR T cells, thus providing proof of concept for the use of this cell population as an alternative T cell source for CAR T cell methods and other adoptive cell therapy methods. Building on these discoveries, and other discoveries presented herein, the present invention provides a variety of novel NKG2C+CD8+ T cells, including engineered NKG2C+CD8+ T cells expressing CAR molecules or transgenic T cell receptors, compositions comprising such cells, methods of generating such cells from conventional (NKG2C−) CD8+ T cells, and methods of using such cells, for example in adoptive cell therapy methods.

These and other aspects of the present invention are described further in the Detailed Description, Drawings, Brief Description of the Drawings, Examples and Claims sections of this patent disclosure. Furthermore, one of skill in the art will recognize that the various embodiments of the present invention described throughout this patent disclosure can be combined in various different ways, and that such combinations are within the scope of the present invention.

### BRIEF DESCRIPTION OF THE DRAWINGS

FIG. 1A-F. Prevalence of NKG2C+ CD8 in HCMV seropositive individuals. (FIG. 1A) FACS plots showing the frequency of FcεRIγ-NKG2C+ NK cells and NKG2C+CD8+ T cells in one representative HCMV-seronegative and HCMV-seropositive donor. (FIG. 1B) Pie charts show the prevalence of NKG2C+CD8+ T cells among HCMV-seronegative and HCMV-seropositive individuals according to frequency of the corresponding lymphocyte subset. P-value=<0.0001, was calculated using a χ² test. (FIG. 1C) Graph showing the frequency of NKG2C+CD8+ T cells among HCMV-seronegative (N=119) and HCMV-seropositive individuals (n=212). Statistical analysis was performed by Mann-Whitney test. Dot plots show the minimum and maximum frequency of NKG2C+ cells among total CD8+ T cells found in donors who display a detectable population (above 2% of total CD8+ T cells). (FIG. 1D) graph showing the frequency of NKG2C+CD8+ T cells among individuals homozygous (NKG2C/NKG2C), heterozygous (NKG2C/Neg) or negative (Neg/Neg) for the KLRC2 gene. (FIG. 1E) Venn diagram showing the frequencies of healthy HCMV-seropositive donors (N=137) having FcεRIγ-NKG2C+ NK cells, NKG2C+CD8+ T cells or both lymphocyte subsets. (FIG. 1F) FACS plots showing the frequency of NKG2C+CD8+ T cells present after cord-blood HSCT in two representative patients, experiencing HCMV reactivation or not. Graph on the right show cumulative analysis. Statistical analysis was performed by Mann-Whitney test. ** P-value <0.01; **** P-value <0.0001.

FIG. 2A-C. Phenotype of NKG2C+CD8+ T cells. (FIG. 2A) Dot plots showing CD56, CD4 and CD8 expression on NKG2C+ cells or NKG2C− cells. Graphs on the right show cumulative analysis from 88 donors. Statistical significance was calculated using student-t test. (FIG. 2B) Left: UMAP plots showing the cumulative expression of NKG2C, NKG2A and KIR receptors in total CD8+ T cells from 88 donors, Right: Graph showing the frequency of NKG2C+CD8+ T cells expressing the indicated KIR receptors compared to NKG2C−CD8+ T cells or NKG2A+CD8+ T cells, segregated by donor-expressed KIR ligands HLA-C1, -C2, -Bw4 and Bw6. Statistical analysis was performed by student-t test comparing the NKG2C−CD8+ or the NKG2A+CD8+ vs NKG2C+CD8+ for each KIR within the indicated HLA haplotype. (FIG. 2C) Left: FACS histograms show staining of the indicated markers in one representative individual, Right: UMAP plots show the expression of the indicated markers in total CD8+ T cells, cumulative of 6 different individuals. ** P-value <0.01; **** P-value <0.0001.

FIG. 3A-C. NKG2C+CD8+ T cells are PD-1 negative at rest and do not upregulate PD-1 even after strong stimulation. (FIG. 3A) NKG2C− and NKG2C+CD8+ T cells from the same representative HCMV-seropositive donor were stained for PD-1 surface expression at rest. (FIG. 3B) Cumulative analysis for PD-1 expression on NKG2C−CD8+ and NKG2C+CD8+ T cells in 88 HCMV-seropositive donors. Statistical analysis was performed by student-t test comparing the NKG2C− vs NKG2C+CD8+ within the same individuals. (FIG. 3C) Left: Total CD8+ T cells were CTV labelled and stimulated for 7 days with beads against CD3/CD2/CD28 and proliferation indexes were analyzed separately for NKG2C− and NKG2C+CD8+ T cells monitoring CTV dilutions, Right: PD-1 expression was analyzed on every mitotic generation following 7 days stimulation with beads against CD3/CD2/CD28 in 3 independent donors. **** P-value <0.0001.

FIG. 4A-G. NKG2C+ CD8 T cells acquire NK-cell transcriptional features and downregulate a BCL11B-dependent transcriptional program. NKG2A−NKG2C+ (NKG2C+CD8+) or NKG2A−NKG2C− (DN) CD8+ T cells were sorted from PBMC and processed for RNA sequencing. (FIG. 4A) Volcano plot depicts log 2 fold change on x-axis and −log 10(p-value) (calculated by Wald tests) on y-axis. Dots highlight DE genes when comparing NKG2C+ versus DN CD8+ T cells. Highlighted are top 10 DE genes ranked by FDR-corrected p-value. Dotted vertical and horizontal lines mark +/−1 and p=0.05, respectively. (FIG. 4B) Bar plots display −log 10 (p-value) of gene set enrichment in genes higher in DN (gray) or higher in NKG2C+ (black) CD8+ T cells. Fractions within bar plots indicate number of DE genes found within the total gene set. (FIG. 4C) Heatmaps show expression of curated NK-cell identity genes (top) and T-cell identity genes (bottom). Asterisks indicate genes found within the “Natural killer cell mediated cytotoxicity” gene set displayed in (FIG. 4B). (FIG. 4D) Heatmap shows expression of top 20 DE genes that encode transcription factors. (FIG. 4E) Bar plots display −log 10 (p-value) calculated by hypergeometric tests evaluating overrepresentation of BCL11B-dependent genes among NKG2C+ versus DN DE genes. Tests were performed using any combination of gene sets that were either upregulated or downregulated in either comparison, as represented by Venn diagrams. Absolute numbers of their differences and intersection are indicated. (FIG. 4F) Heatmap shows expression of BCL11B-dependent genes that are also DE in NKG2C+ versus DN comparison. Of the genes listed, LYN and those listed above LYN are upregulated upon BCL11B-deficiency, while SYDE2 and the genes listed below SYDE2 are downregulated. (FIG. 4G) Histograms show instracellular staining for BCL11B in three different donors, gated on total NK cells, DN or NKG2C+CD8+ T cells. Dotted histograms represent isotype controls. DE=differentially expressed; FDR=false discovery rate.

FIG. 5A-G. NKG2C+CD8+ T cells can be generated in vitro by BCL11B deletion. (FIG. 5A) FACS histograms showing BCL11B intracellular expression on CD8+ T cells that had been electroporated with Cas/9 and control mRNA guides or BCL11B specific mRNA guides. (FIG. 5B) FACS plots showing the frequency of CD8+CD56+ cells on the control and BCL11B-KO cells after 2 or 4 weeks of expansion with CD3/CD2/CD28 beads or K562 mbIL-21 HLA-E:VMAPRTLFL (SEQ ID NO. 1). Graph on the right shows cumulative analysis from 2 independent donors (FIG. 5C) FACS plots showing the frequency of CD45RA+CCR7−CD8+ cells on the control and BCL11B-KO cells after 2 or 4 weeks of expansion with CD3/CD2/CD28 beads or K562 mbIL21 HLA-E:VMAPRTLFL (SEQ ID NO. 1). Graph on the right shows cumulative analysis from 2 independent donors. (FIG. 5D) Cumulative analysis from 2 donors on PD1+CD8+ T cells in control or BCL11B-KO cells after 2 or 4 weeks of expansion with CD3/CD2/CD28 beads or K562 mbIL-21 HLA-E:VMAPRTLFL (SEQ ID NO. 1). (FIG. 5E) FACS plots showing the frequency of NKG2A+ vs NKG2C+ and CD56+DAP12+CD8 T cells on the control and BCL11B-KO cells after 4 weeks of expansion with K562 mbIL-21 HLA-E:VMAPRTLFL (SEQ ID NO. 1). (FIG. 5F) Cumulative analysis of two independent donors from the data shown in (FIG. 5E). (FIG. 5G) Representative flow cytometry plots showing degranulation (CD107a) and intracellular IFN-γ expression by K562 mbIL-21 HLA-E:VMAPRTLFL (SEQ ID NO. 1) expanded pre-gated NKG2C+ or NKG2C−CD8+ T cells after 6 hours stimulation with the indicated target cells.

FIG. 6A-F. NKG2C+CD8+ T cells express functional NKG2C that can cooperate with TCR activation in degranulation and IFN-γ production. (FIG. 6A) Total PBMC cells were stimulated with the indicated plate-bound mAbs for 6 h. Representative flow cytometry plots showing degranulation (CD107a) and intracellular IFN-γ expression by pre-gated NKG2C+ or NKG2C−CD8+ T cells after triggering with plate-bound mAbs (10 μg/mL). (FIG. 6B) Graphs showing the percentage of CD107a+ and IFN-γ+ cells from 7 independent donors. Data are shown as mean±SD. Statistical analysis was performed by Mann-Whitney test comparing the NKG2C−CD8+ vs NKG2C+ CD8+ for each antibody. (FIG. 6C) FACS plots representing CD107a and intracellular IFN-γ expression by NKG2C− or NKG2C+CD8+ T cells after anti-CD3 (1 μg/mL) plate-bound stimulation. (FIG. 6D) Corresponding graphs showing the percentage of CD107a+ and IFN-γ+ cells from 7 independent donors. Data are shown as mean±SD. (FIG. 6E) Representative FACS plots showing the percentage of CD107a+ cells after triggering of NKG2C+CD8+ T cells with plate-bound anti-CD3 or anti-NKG2C mAb alone or in combination. (FIG. 6F) Data are shown as the mean±SD for 7 independent donors. Statistical significance was calculated by ANOVA with the Friedman test and Dunn's post-test correction. ***P-value ≤0.001, ** P-value <0.01, * P-value ≤0.05.

FIG. 7A-L. NKG2C+CD8+ T cells potent anti-tumor and anti-HCMV effector functions are mediated my their NKG2C and TCR specificity. (FIG. 7A) Representative flow cytometry plots showing degranulation (CD107a) and intracellular IFN-γ expression by pre-gated NKG2C+ or NKG2C−CD8+ T cells after 6 hours stimulation with K562 HLA-E:VMAPRTLFL (SEQ ID NO. 1) target cells. (FIG. 7B) Graph showing cumulative analysis of CD107a+ and IFN-γ+ NKG2C+ or NKG2C−CD8+ T cells from 12 independent donors against K562 HLA-E:VMAPRTLFL (SEQ ID NO. 1) target cells. Statistical significance was calculated using Mann-Whitney test. (FIG. 7C) In vitro cytotoxicity of FACS-sorted NKG2C+ or NKG2C−CD8+ T cells against K562 HLA-E:VMAPRTLFL (SEQ ID NO. 1) target cells was assessed using a 6 hours bioluminescence assay. Results from 3 independent donors are shown. Statistic was calculated using a 2-way ANOVA comparing the mean of each E:T ratio between NKG2C− and NKG2C+CD8+ T cells. (FIG. 7D) Representative flow cytometry plots showing degranulation (CD107a) and intracellular IFN-γ expression by pre-gated NKG2C+CD8+ T cells against K562 HLA-E:VMAPRTLFL (SEQ ID NO. 1) in the presence of the indicated blocking mAbs (10 μg/mL). (FIG. 7E) Graph showing cumulative analysis of 4 independent donors from the experiment shown in (FIG. 7D). Statistical significance was calculated by ANOVA with the Friedman test and Dunn's post-test correction. (FIG. 7F) CD8+ T cell from 6 healthy donor PBMC were stained with a combination of different monoclonal antibodies against individual VP TCRs and analyzed by flow cytometry (IOTest Beta Mark kit, Beckman Coulter). Cells were pre-gated on NKG2A+NKG2C− (NKG2A+), NKG2A−NKG2C+ (NKG2C+) and NKG2A−NKG2C− CD8+ T cells. (FIG. 7G) In vitro cytotoxicity of FACS-sorted NKG2C+ or NKG2C−CD8+ T cells against the indicated AML target cell lines was assessed using a 6 hours bioluminescence assay. Results from 3 independent donors are shown. Statistic was calculated using a 2-way ANOVA comparing the mean of each E:T ratio between NKG2C− and NKG2C+CD8+ T cells. (FIG. 7H) Representative flow cytometry plots showing degranulation (CD107a) and intracellular IFN-γ expression by pre-gated NKG2C+ or NKG2C−CD8+ T cells after 6 hours stimulation with uninfected or HCMV-infected human fibroblasts. (FIG. 7I) Cumulative analysis of CD107a+ and IFN-γ+ NKG2C+ or NKG2C−CD8+ T cells from 12 independent donors against uninfected or HCMV-infected human fibroblasts. Statistical significance was calculated using Mann-Whitney test. (FIG. 7J) FACS plots showing TCRαβ expression on NKG2C+CD8+ or NKG2C−CD8+ T cells that had been electroporated with Cas/9 and TRAC specific mRNA guides. (FIG. 7K) Cumulative analysis of CD107a+ and IFN-γ+ cells against the indicated targets of the three independent donors shown in (FIG. 7J). Statistical significance was calculated by ANOVA with the Friedman test and Dunn's post-test correction. (FIG. 7L) NKG2C+CD8+ T cells from 5 donors were stained with the indicated HLA:E tetramers in the presence of a CD94 blocking antibody. ****P-value ≤0.0001, ***P-value ≤0.001, **P-value ≤0.01, *P-value ≤0.05.

FIG. 8A-F. NKG2C+CD8+ T cells proliferate in response to HLA-E and display superior tumor killing if transduced with a CD19-CAR. (FIG. 8A) Left: Representative plots showing total PBMC cells stimulated for 7 days with the indicated stimuli and pre-gated on CD56-CD3+CD8+ cells, Right: Cumulative analysis from 3 independent donors. Statistical significance was calculated by ANOVA with the Friedman test and Dunn's post-test correction. (FIG. 8B) Total cell counts of pre-sorted NKG2C+CD8+ T cells stimulated with K562 mb-IL21 HLA-E:VMAPRTLFL (SEQ ID NO. 1) (1:1) for the indicated number of days. (FIG. 8C) Total CD8+ T cells were CTV labelled and exposed for 7 days to the indicated stimuli. Pre-gated NKG2C+ or NKG2C−CD8+ T cells were then analyzed for proliferation monitoring CTV dilutions. Statistical analysis was calculated using a 2-way ANOVA. (FIG. 8D) NKG2C− and NKG2C+CD8+ T cells were FACS sorted and left untransduced or transduced with a 1928z CD19-targeting CAR construct. Cytotoxicity was assessed using a standard 51Cr assay after 24 h of exposure to the CD19+ cell line NALM6. (FIG. 8E) PD1 surface expression on NKG2C− CD8 and NKG2C+ CD8 cells transduced with the 1928z CD19-targeting CAR construct. (FIG. 8F) Histogram shows HLA-E surface staining on NALM-6 cells. ****P-value ≤0.0001, ***P-value ≤0.001, **P-value ≤0.01, *P-value ≤0.05.

FIG. 9A-C. NKG2C+CD8+ T cells do not expand with age and are not specific for the most immunogenic HCMV peptides. (FIG. 9A) Left: Correlation between frequency of FcεRIγ− NKG2C+ NK cells and frequency of NKG2C+CD8+ T cells. Right: Correlation between frequency of NKG2C+CD8+ T cells and age of the donor tested. (FIG. 9B) FACS plots showing staining of HCMV-specific CD8+ T cells with HLA class I matched HCMV-pp65 tetramer in two donors with the indicated HLA backgrounds. (FIG. 9C) FACS plots intracellular IFN-γ expression by pre-gated NKG2C+ or NKG2C−CD8+ T cells after 16 hours stimulation with PMA/Ionomycin (P+I) or the indicated HCMV peptides.

FIG. 10A-D. T cell differentiation markers analysis on HCMV-seronegative donors and comparison with NK cells. (FIG. 10A) FACS histograms show staining of the indicated markers in one representative HCMV-seronegative individual. (FIG. 10B) Cumulative analysis of the indicated markers on pre-gated NKG2A−NKG2C− (DN), NKG2A+NKG2C− (NKG2A+) or NKG2A−NKG2C+(NKG2C+) CD8+ T cells from 4 different HCMV-seronegative individuals. Statistical significance was calculated by ANOVA with the Friedman test and Dunn's post-test correction. (FIG. 10C) FACS histograms show staining of the indicated markers in one representative HCMV-seropositive individual. *P-value ≤0.05. (FIG. 10D) FACS plots from one representative donor showing the frequency of KIR2DL1/S1, KIR2DL2/L3/S2 and KIR3DL1 on NK cells, naïve (CD45RA+CCR7+), NKG2C− TEMRA (CD45RA+CCR7−) or NKG2C+CD8+ T cells. Graphs on the right show cumulative analysis from 5 independent donors. Statistical significance was calculated by ANOVA with the Friedman test and Dunn's post-test correction. ****P-value ≤0.0001, ***P-value ≤0.001, **P-value ≤0.01, *P-value ≤0.05.

FIG. 11A-D. NKG2C+CD8+ T cells are transcriptionally distinct from TEMRA cells. (FIG. 11A) Heatmap showing expression of genes that are a part of the NK signature (380 out of 1569) in NKG2C+CD8+ T cells vs DN CD8+ T cells. (FIG. 11B) Heatmap showing expression of genes that are a part of the TEMRA signature (131 out of 859) in NKG2C+CD8+ T cells vs DN CD8+ T cells. (FIG. 11C) Empirical cumulative distribution function (ecdf) of the log 2[fold change] in gene expression between DN CD8+ T cells versus NKG2C+CD8+ T cells for all NK cell signature genes (red) and TEMRA signature genes (blue) compared to all genes (black). (FIG. 11D) Histograms show intracellular staining for BCL11B in one representative donor, gated on total NK cells, NKG2C+CD8+ T cells, DN TEMRA (CD45RA+CCR7−) or naïve (CD45RA+CCR7+) cells. Dotted histograms represent isotype controls. Graph on the right shows cumulative analysis from 5 independent donors. Statistical significance was calculated by ANOVA with the Friedman test and Dunn's post-test correction. ****P-value ≤0.0001, * P-value ≤0.05.

FIG. 12A-B. NKG2C+CD8+ T cells showed the narrowest TCR Vβ chain usage and those from HCMV-seronegative donors are polyclonal for their TCR Vβ chain repertoire. (FIG. 12A) CD8+ T cells from donor 1 and donor 5 were pre-gated on naïve (CD45RA+, CCR7+), TEMRA (CD45RA+, CCR7−) NKG2A−NKG2C−, TEMRA NKG2A+NKG2C− and TEMRA NKG2A−NKG2C+ and analyzed by flow cytometry, with the IOTest Beta Mark kit, for individual Vβ TCRs distribution. (FIG. 12B) CD8+ T cells from 3 HCMV-seronegative healthy donor PBMC were stained with a combination of different monoclonal antibodies against individual Vβ TCRs and analyzed by flow cytometry. Cells were pre-gated on NKG2A+NKG2C− (NKG2A+), NKG2A−NKG2C+(NKG2C+) and NKG2A−NKG2C− CD8+ T cells.

FIG. 13A-F. Functional characterization of NKG2C+CD8+ T cells. (FIG. 13A) Graphs showing cumulative analysis from 3 independent donors of degranulation (CD107a) and intracellular IFN-γ expression by pre-gated NKG2C−CD8+ or NKG2C+CD8+ T cells against the indicated targets in the presence of CD94 blocking mAb (10 μg/mL). Statistical significance was calculated by ANOVA with the Friedman test and Dunn's post-test correction. (FIG. 13B) HLA-E surface staining of the indicated AML cell lines. (FIG. 13C) Graphs showing cumulative analysis from 3 independent donors of degranulation (CD107a) and intracellular IFN-γ expression by pre-gated NKG2C+CD8+ T cells, NKG2C− TEMRA (CD45RA+CCR7−) or naïve (CD45RA+CCR7+) CD8+ T cells against the indicated targets. Statistical significance was calculated by ANOVA with the Friedman test and Dunn's post-test correction. (FIG. 13D) Graphs showing cumulative analysis from 5 independent donors of degranulation (CD107a) and intracellular IFN-γ expression by pre-gated NKG2C+CD8+ T cells or NK cells (gated on CD56+ CD3−) against the indicated targets. Statistical analysis was performed by student-t test comparing the functional response of NKG2C+CD8+ against 721.221 vs 721.221-Cw*3 or NK cells against 721.221 vs 721.221-Cw*3 within the same individuals. (FIG. 13E) Histogram shows HLA-E surface staining on 721.221 wild type cells. (FIG. 13F) Graphs showing cumulative analysis from 3 independent donors of degranulation (CD107a) and intracellular IFN-γ expression by pre-gated NKG2C+CD8+ T cells against the indicated targets. Statistical analysis was performed by student-t test. ****P-value ≤0.0001, ***P-value ≤0.001, **P-value ≤0.01, *P-value ≤0.05.

FIG. 14A-D. Phenotype and function of NKG2C+CD8+ T cells after one week expansion with CD3/CD2/CD28 beads or K562 HLA-E:VMAPRTLFL (SEQ ID NO. 1) mbIl-21. (FIG. 14A) FACS histograms show staining of the indicated markers in one representative donor at baseline (prior any stimulation) or after 7 days stimulation with K562 HLA-E:VMAPRTLFL (SEQ ID NO. 1) mbIl-21 or CD3/CD2/CD28 beads. Cells were pre-gated on NKG2A− NKG2C− or NKG2A−NKG2C+ CD8+ T cells. (FIG. 14B) Cumulative analysis from 3 independent donors for the indicated markers and stimuli. Cells were pre-gated on NKG2A− NKG2C− or NKG2A−NKG2C+ CD8+ T cells. (FIG. 14C) Representative flow cytometry plots showing degranulation (CD107a) and intracellular IFN-γ expression by pre-gated NKG2C+ or NKG2C−CD8+ T cells after 6 hours stimulation with the indicated target cells. (FIG. 14D) Cumulative analysis of CD107a+ and IFN-γ+NKG2C+ or NKG2C− CD8+ T cells from 3 independent donors against the indicated targets. Statistical significance was calculated using Mann-Whitney test comparing the NKG2C+ versus the NKG2C−NCD8+ T cell population against the same target. ****P-value ≤0.0001, **P-value ≤0.01.

### DETAILED DESCRIPTION

The practice of the present invention will employ, unless otherwise indicated, conventional techniques of cell biology, cell culture, molecular biology, transgenic biology, microbiology, recombinant DNA technology, and immunology, which are within the skill of the art. Such techniques are explained fully in the literature. See, e.g., Ausubel et al. eds. (2015) Current Protocols in Molecular Biology (John Wiley and Sons); Greenfield, ed. (2013) Antibodies: A Laboratory Manual (2nd ed., Cold Spring Harbor Press); Green and Sambrook, eds. (2012), Molecular Cloning: A Laboratory Manual (4th ed., Cold Spring Harbor Laboratory Press); Krebs et al., eds. (2012) Lewin's Genes XI (11th ed., Jones & Bartlett Learning); Freshney (2010) Culture Of Animal Cells (6th ed., Wiley); Weir and Blackwell, eds., (1996) Handbook Of Experimental Immunology, Volumes I-IV (5th ed., Wiley-Blackwell); Borrebaeck, ed. (1995) Antibody Engineering (2nd ed., Oxford Univ. Press); Glover and Hames, eds., (1995) DNA Cloning: A Practical Approach, Volumes I and II (2nd ed., IRL Press); Rees et al., eds. (1993) Protein Engineering: A Practical Approach (1st ed., tabl IRL Press); Mayer and Walker, eds. (1987) Immunochemical Methods In Cell And Molecular Biology (Academic Press, London); Nisonoff (1984) Introduction to Molecular Immunology (2nd ed., Sinauer Associates, Inc.); and Steward (1984) Antibodies: Their Structure and Function (1st ed., Springer Netherlands).

Unless defined otherwise, all technical and scientific terms used herein have the same meaning as commonly understood by one of ordinary skill in the art to which this invention is related. For example, The Dictionary of Cell and Molecular Biology (5th ed. J. M. Lackie ed., 2013), the Oxford Dictionary of Biochemistry and Molecular Biology (2d ed. R. Cammack et al. eds., 2008), and The Concise Dictionary of Biomedicine and Molecular Biology (2d ed. P-S. Juo, 2002) can provide one of skill with general definitions of some terms used herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form. Numeric ranges provided herein are inclusive of the numbers defining the range.

Where a numeric term is preceded by “about” or “approximately,” the term includes the stated number and values±10% of the stated number.

Numbers in parentheses or superscript following text in this patent disclosure refer to the numbered references provided in the “Reference List” section of this patent disclosure.

Wherever embodiments are described with the language “comprising,” otherwise analogous embodiments described in terms of “consisting of” and/or “consisting essentially of” are included.

As used herein a “subject” is any individual for whom diagnosis, prognosis, or therapy is desired. In some embodiments the subject is a mammalian subject. In some embodiments the subject is a human subject.

The term “composition” refers to a preparation that is in such form as to permit the biological activity of the cells contained therein and which contains no additional components that are unacceptably toxic to a cell or subject to which the composition may be administered. Such compositions can be sterile.

As used herein, the terms “treat,” “treating,” and “treatment” encompass achieving, and/or performing a method that achieves, a detectable improvement in one or more clinical indicators or symptoms associated with the condition being treated. For example, in the case of treatment of a tumor such terms include, but are not limited to, reducing the rate of growth of the tumor (or tumor cells), halting the growth of the tumor (or tumor cells), causing regression of the tumor (or tumor cells), reducing the size of the tumor (for example as measured in terms of tumor volume or tumor mass), reducing the grade of the tumor, eliminating the tumor (or tumor cells), preventing, delaying, or slowing recurrence (rebound) of the tumor, improving symptoms associated with the tumor, improving survival from the tumor, inhibiting or reducing spreading of the tumor or tumor cells (e.g. metastases), and the like. Similarly, as used herein, the terms “treat,” “treating,” and “treatment” encompass achieving, and/or performing a method that achieves, a detectable enhancement or detectable stimulation of an anti-tumor immune response in a subject.

The terms “tumor” and “cancer” may be used interchangeably herein.

Other terms are defined elsewhere in this patent disclosure, or else are used in accordance with their usual meaning in the art.

### T Cells

The present invention involves human T cells. Human T cells can be obtained readily from human subjects, for example from peripheral blood samples, using standard methods known in the art, including, but not limited to, the methods described in the Examples section of this patent disclosure.

In general, the T cells of the present invention express the proteins (sometimes referred to as markers) NKG2C and CD8, i.e., they are NKG2C+ CD8+ T cells. In some embodiments the T cells express, or do not express, certain other specified markers. For example, in many embodiments the T cells of the present invention express NKG2C and CD8 but do not express NKG2A, i.e., they are NKG2C+ NKG2A− CD8+ T cells.

For each of the embodiments provided herein where the T cells are referred to as being positive (or “+”) for a specified marker, the present invention also includes alternative and otherwise analogous embodiments where the T cells express high (“hi” or “high”) levels of such markers. Similarly, for each of the embodiments provided herein where the T cells are referred to as being negative (or “−”) for a specified marker, the present invention also includes alternative and otherwise analogous embodiments where the T cells express low (“lo” or “low”) levels of such markers. The “positive” versus “negative” and “hi” versus “lo” terminology is routinely used and accepted in the T cell art as a way of characterizing T cell marker profiles. One or ordinary skill in the art can readily determine if a cell is positive or negative or high or low for a given marker using standard techniques and analysis used in the T cell art, including flow cytometry and cell sorting methods. For example, it is standard and accepted to practice to determine whether a cell is positive or negative or high or low for a given marker based on analysis of flow cytometry data (for example using scatter plots), and/or based on the ability to obtain such cells by cell sorting, and/or by using appropriate gating methods and parameters in flow cytometry and/or cell sorting methods.

In some embodiments the T cells of the present invention are NKG2C+ CD8+ T cells. In some embodiments the T cells are NKG2C+ NKG2A− CD8+ T cells.

In some embodiments the T cells are CD45RA+ CD45RO− T cells. In some embodiments the T cells are CD45RA− CD45RO+. In some embodiments the T cells are activated T cells and are CD45RA− CD45RO+. Importantly, it has been demonstrated in the Examples section of this patent disclosure, that prolonged expansion and/or activation NKG2C+ CD8+ CD45RA+ CD45RO− T cells ex vivo leads to generation of NKG2C+ CD8+ CD45RA− CD45RO+ T cell populations.

In some embodiments the T cells of the present invention are CCR7−.

In some embodiments the T cells of the present invention are KIRs+.

In some embodiments the T cells of the present invention are BCL11B− or BCL11B− lo.

In some embodiments the T cells of the present invention are PD1− or PD1lo.

In some embodiments the T cells of the present invention are CD45RA+ CD45RO− CCR7− KIRs+ BCL11B−.

In some embodiments the T cells of the present invention are CD45RA+ CD45RO− CCR7− KIRs+ PD1−.

In some embodiments the T cells of the present invention are activated and are CD45RA− CD45RO+ CCR7− KIRs+ BCL11B−.

In some embodiments the T cells of the present invention are activated and are CD45RA− CD45RO+ CCR7− KIRs+ BCL11B− PD1−.

In some embodiments the T cells of the present invention are positive for one or more markers selected from the group consisting of: KIR2DS4, SYK, ITGAX, KIR2DL3, S1PR5, PRF1, TBX21, NCR1, GZMB, GNLY, IL2RB, KIR2DL1, KIR2DL4, KIR3DL2, KLRC2, KLRC3, TYROBP, CD244, KLRC1, LAT2, KIR3DL1, PLCG2, FCGR3A, LYN, NCAM1, SLAMF7, JAZF1, SOX13, IKZF2, TBX21, ASCL2, L3MBTL4, HOPX, CD56, KIR, CD94, DAP12+ and TCRD□□. In some embodiments the T cells of the present invention are positive for all of these listed markers.

In some embodiments the T cells of the present invention are positive for one or more markers selected from the group consisting of: KLRC1, LAT2, KLRC2, TYROBP, PDE4A, CCDC50, NCR1, LYN, PIK3AP1, “MGARP, NDUFC1”, SERPINB6, RAB3D, ANKRD53, “KLRC3, RP11-277P12.6”, RGS9, KIR2DL4, C9orf66, IGFBP7, GAS7, VAV3, RIN3, JAZF1, SGSM1, SOX13, IKZF2, CD244, SRGAP1, FUCA2, SLFN13, FAM13A, AC005562.1, GOLGA8N, LRRC63, PTGDR, TRG-AS1, GOLIM4, PSMD1, INPP1, SPRYD7, NAGS, B3GNT7, “RHOC, RP11-426L16.10”, KIR3DL2, CEP78, SLC43A2, IMPG2, ADAMTS14, CD63, FCGR3A, VANGLI, GPR153, WDR41, HEATR3, PIF1, RP11-428G5.5, IFI30, ITGAX, MTSS1, DUSP5, CTD-2383M3.1, KRT5, SWAP70, ADAM8, ANXA4, B9D1, NHLRC2, DRAXIN, OSBPL5, TTC38, “WDR66, RP11-87C12.2”, RP11-169F17.1, GLB1L2, CCDC102A, APOBR, AC005235.1, RHOQ, TBX21, TNFRSF21, CPNE8, LIM2, ABHD15, CD300LB, BSPRY, NCALD, NCAM1, ARHGEF12, ANO10, NAPB, CATSPER1, AKAP5, SPIRE1, RCBTB2, YPEL1, PLEKHA6, AANAT, C17orf58, SLC25A30, ARRB1, KLRF1, and PLA2G16. In some embodiments the T cells of the present invention are positive for all of these listed markers.

In some embodiments the T cells of the present invention are negative for one or more markers selected from the group consisting of: TCF7, CD27, CD5, PDCD1, CD28, CCR7, CD6, IL7R, THEMIS, TESPA1, BCL11B, ZNF518B, SATB1, ZEB1, SREBF1, BACH2, FOXP1, YBX3, ARID5A, ZFP36L1, RERE, E2F3, KLF7, and PD1. In some embodiments the T cells of the present invention are negative for all of these listed markers.

In some embodiments the T cells of the present invention are negative for one or more markers selected from the group consisting of: CD28, SERINC5, PAGI, RCAN3, BCL11B, CD6, RAB30, C1orf228, PASK, ITM2C, ZNF518B, OXNAD1, TNFAIP8, TGFBR2, PLK3, SATB1, TNFSF8, LIMK2, AQP3, TRABD2A, SFRP5, FAM102A, SNN, ZEB1, ABCG1, PABPC1, DGKA, “RNF125, RP11-53I6.2”, RBSN, SLC2A3, PTGER4, FAM134B, MPZL3, SPINT2, GRAMD1A, TNFRSF25, SNX9, SARAF, SFXN1, JMJD1C, SREBF1, S100B, SGSM2, CERK, SEC14L2, TESPA1, LRIG1, YIPF5, GIMAPS, JOSD1, AMICA1, TACC3, PCSK1N, BACH2, CCR7, ACTN1, RPLP0, FOXP1, VIPR1, SETD1B, RP5-1187M17.10, CCDC57, DSC1, ATHL1, DDX21, LTB, IL7R, POLR3E, SH2D3A, THEMIS, SLC8B1, PFKFB3, “FLT3LG, CTD-3148I10.9, CTD-3148I10.15”, LAPTM5, PIK3IP1, GAS5, YBX3, STMN3, PELI1, RBM12, GPR183, PIK3IP1-AS1, ITPKB, PIGA, PDCD1, RGS10, “RAPGEF6, CTC-432M15.3”, ASXL1, RHOH, TOB1, CD248, LDLRAP1, NT5E, APOL3, CXCL16, NOP58, RRM1, EEF2, TSPYL2, APBA2. In some embodiments the T cells of the present invention are negative for all of these listed markers.

In some embodiments, T cells having the specified marker profiles described herein can be obtained from mixed T cell populations (such as those obtained from human subjects) using standard methods known in the art such as flow cytometry and/or fluorescence activated cell sorting based methods. For example, in some embodiments, T cells positive for a specified marker protein (e.g., NKG2C) can be obtained from mixed populations of T cells (i.e., a population of T cells containing cells that express the specified marker and cells that do not express the specified marker) by contacting the cells with antibody that binds to the marker protein and performing fluorescence activated cell sorting to obtain cells bound by the antibody.

In some embodiments, T cells having specified marker profiles can be generated by expressing recombinant nucleic acid molecules encoding the specified marker proteins in the T cells (to created marker+cells) and/or by inhibiting expression of or knocking out genes encoding the specified marker proteins in the T cells (to generate marker-cells). It should be noted that as used herein, “knocking out” a gene includes disrupting or perturbing a gene to the extent that it is functionally knocked out such that expression/production of the protein encoded by the gene is prevented, even if portions of the gene remain intact.

In some embodiments, NKG2C+ CD8+ T cells can be obtained by knocking out BCL11B expression in conventional NKG2C− CD8+ T cells (i.e., by generating NKG2C− BCL11B− CD8+ T cells). As demonstrated in the Examples section of this patent disclosure, after knocking out the BCL11B gene the cells express NKG2C i.e., they become NKG2C+ BCL11B− CD8+ T cells (which may also be referred to herein as NKG2C+ CD8+ BCL11B KO cells). Thus, in one embodiment the present invention provides NKG2C− BCL11B− CD8+ T cells, while in another embodiment the present invention provides NKG2C+ BCL11B− CD8+ T cells. Similarly, in some embodiments, the present invention provides methods for producing NKG2C+ CD8+ T cells from NKG2C− CD8+ T cells, such methods comprising inhibiting the expression of or knocking out the BCL11B gene in NKG2C− CD8+ T cells.

In some embodiments the T cells of the present invention comprise a T cell receptor (TCR) comprising a TRBV-14 Vβ chain.

In some embodiments the T cells of the present invention comprise a T cell receptor (TCR) comprising a TRBV-28 Vβ chain.

In some embodiments the T cells of the present invention are engineered T cells, i.e., T cells that have been genetically modified to express a protein not normally expressed in T cells in nature (i.e., a non-native protein) and/or to inhibit or eliminate (knock out) expression of a protein normally expressed in T cells in nature (i.e., a native protein). Non-native proteins expressed in T cells as a result of such genetic modification may be referred to interchangeably herein as transgenic proteins or engineered proteins or non-native proteins.

Chimeric antigen receptors “CARs”, and T cells that express them (“CAR T cells”) are well known and the terms “CAR” and “CAR T cells” have accepted meanings in the art (see, for example, Feins et al., “An introduction to chimeric antigen receptor (CAR) T-cell immunotherapy for human cancer.” Am. J. Hematol. 2019 May; 94(S1):S3-S9). In brief, chimeric antigen receptors are genetically engineered T cell receptors having an extracellular antigen binding domain that binds to a specific protein (such as a tumor associated antigen), a transmembrane domain, and an intracellular domain having T cell activating functions. In some embodiments the T cells of the present invention are engineered T cells that comprise a recombinant nucleic acid molecule that encodes a chimeric antigen receptor (CAR) and/or that express a CAR—i.e., they are CAR T cells. In some of such embodiments, the CAR comprises an antigen-binding domain that binds to a tumor-associated antigen. For example, in some embodiments the CAR comprises an antigen-binding domain that binds to a tumor-associated antigen selected from the group consisting of CD19, CD20, CD22, CD30, CD33, BCMA, Igk and ROR1. In some embodiments the CAR comprises an antigen-binding domain that binds to the tumor-associated antigen CD19. In some embodiments the CAR comprises an antigen-binding domain that comprises the CDR1, CDR2 and CDR3 domains of the 1928z anti-CD19 CAR. In some embodiments the CAR comprises the antigen-binding domain of the 1928z anti-CD19 CAR. In some embodiments the CAR comprises the 1928z anti-CD19 CAR. The 1928z anti-CD19 CAR and its amino acid sequences is known in the art and is described in published international patent application WO2014/134165 entitled “Compositions and Methods for Immunotherapy,” the contents of which are hereby incorporated by reference.

In some embodiments the T cells of the present invention are engineered T cells that comprise a recombinant nucleic acid molecule that encodes a transgenic T cell receptor (TCR) and/or that express a transgenic TCR. Transgenic TCRs are well known in the art and the term transgenic TCR has an accepted meaning in the art. TCR T-cell therapy involves transducing T cells with a specific TCR that recognizes a desired/known antigen, such as an antigen expressed by tumor cells and presented in the context of MHC molecule on the surface of the tumor cells, which leads to induction of cytotoxic T-cell effector functions and the killing of targeted tumor cells. See, for example, Oppermans et al., “Transgenic T-cell receptor immunotherapy for cancer: building on clinical success.” Therapeutic Advances in Vaccines and Immunotherapy. January 2020. doi:10.1177/2515135520933509, for a review of transgenic T cell receptors and their use in cancer treatment. In some embodiments the T cells of the present invention are engineered T cells that comprise a recombinant nucleic acid molecule that encodes a transgenic T cell receptor (TCR) that recognizes a tumor antigen presented on an MHC molecule.

In some embodiments the T cells of the present invention are engineered T cells in which expression of a native TCR has been inhibited. In some embodiments the T cells of the present invention are engineered T cells in which expression of a native TCR has been eliminated (knocked out).

In some embodiments the T cells of the present invention are expanded ex vivo. Several methods of expanding T cells ex vivo are known, and any such methods can be used, including, but not limited to those methods of expanding T cells described in the Examples section of this patent disclosure.

In some embodiments the T cells of the present invention are activated ex vivo. Several methods of activating T cells ex vivo are known, and any such methods can be used, including, but not limited to those methods of activating T cells described in the Examples section of this patent disclosure.

In some embodiments the T cells of the present invention may be administered to human subjects. In some of such embodiments the T cells are autologous with respect to the subject to whom they may be administered. In some of such embodiments the T cells are allogeneic with respect to the subject(s) to whom they may be administered.

In some embodiments the T cells of the present invention may be used in any of the methods described herein.

### Compositions

In some embodiments the present invention provides compositions that comprise the T cells, or populations of T cells, of the present invention. Such compositions comprise the T cells of the present invention and at least one additional component (“excipient”). Examples of such excipients include, but are not limited to, diluents, saline solutions, cell culture media, buffers, carriers, stabilizers, antibiotics, preservatives, and the like. In some embodiments the compositions are sterile.

In some embodiments at least 70% of the cells in the composition are the T cells of the invention. In some embodiments at least 75% of the cells in the composition are the T cells of the invention. In some embodiments at least 80% of the cells in the composition are the T cells of the invention. In some embodiments at least 85% of the cells in the composition are the T cells of the invention. In some embodiments at least 90% of the cells in the composition are the T cells of the invention. In some embodiments at least 95% of the cells in the composition are the T cells of the invention. In some embodiments at least 98% of the cells in the composition are the T cells of the invention. In some embodiments at least 99% of the cells in the composition are the T cells of the invention. In some embodiments 100% of the cells in the composition are the T cells of the invention.

### Methods of Use

The present invention also provides various methods of use of the T cells and compositions described herein.

In some embodiments the present invention provides methods for killing tumor cells, the methods comprising contacting tumor cells with T cells as described herein, or compositions comprising such T cells. In some of such embodiments the tumor cells are in vitro. In some of such embodiments the tumor cells are in vivo.

In some embodiments the present invention provides methods for enhancing or stimulating an anti-tumor immune response in a subject, the methods comprising administering T cells as described herein, or compositions comprising such T cells, to a subject in need thereof.

In some embodiments the present invention provides methods for treating a tumor in a subject, the methods comprising administering T cells as described herein, or compositions comprising such T cells, to a subject in need thereof.

In some embodiments the present invention provides methods for treating a viral infection in subject, the methods comprising administering T cells as described herein, or compositions comprising such T cells, to a subject in need thereof.

Those methods described above and/or elsewhere herein that involve administering the T cells of the present invention, or compositions comprising such cells, to subjects, may be referred to as adoptive cell therapy methods.

In some embodiments the present invention provides adoptive cell therapy methods that comprise: (a) expanding and/or activating T cells having marker profiles as described herein ex vivo, wherein the T cells are obtained from a donor subject, and (b) administering the expanded and/or activated T cells having marker profiles as described herein to a recipient subject.

In some embodiments the present invention provides adoptive cell therapy methods that comprise: (a) obtaining a population of T cells from a donor subject, (b) expanding and/or activating T cells having marker profiles as described herein that are present in the population of T cells ex vivo, and (c) administering the expanded and/or activated T cells having marker profiles as described herein to a recipient subject.

In some embodiments the present invention provides adoptive cell therapy methods that comprise: (a) obtaining a mixed population of T cells from a donor subject, (b) isolating T cells having marker profiles as described herein from the mixed population of T cells, (c) expanding and/or activating the T cells having the marker profiles as described herein ex vivo, and (d) administering the expanded and/or activated T cells having the marker profiles as described herein to a recipient subject.

In some of such embodiments, prior to the step of administering the T cells having a marker profile as described herein to the recipient subject, the cells are genetically modified ex vivo, for example to introduce a nucleic acid molecule encoding a chimeric antigen receptor or transgenic T cell receptor as described herein

In some of such embodiments, the donor subject and the recipient subject are the same individual—i.e., the method is an autologous adoptive cell therapy method and the T cells that are administered to the subject are autologous with respect to the subject.

In some of such embodiments, the donor subject and the recipient subject are different individuals of the same species—i.e., the method is an allogeneic adoptive cell therapy method and the T cells that are administered to the subject are allogeneic with respect to the subject.

In some embodiments the subjects are mammalian subjects. In some embodiments the subjects are human subjects.

In those methods relating to tumors (or cells from tumors, i.e., tumor cells), in some embodiments the tumors are solid tumors. In some embodiments the tumors are circulating tumors. In some embodiments the tumors are hematologic tumors. In some embodiments the tumor is a lymphoma. In some embodiments the tumor is a leukemia. In some embodiments the tumor is a myeloma. In some embodiments the tumor is an acute lymphoblastic leukemia (ALL). In some embodiments the tumor is a B-cell acute lymphoblastic leukemia. In some embodiments the tumor is an acute myelogenous leukemia (AML). In some embodiments the tumor is a B-cell non-Hodgkin lymphoma (NHL). In some embodiments the tumor is a follicular lymphoma (FL). In some embodiments the tumor is a multiple myeloma. In some embodiments the tumor is a diffuse large B cell lymphoma (DLBCL). In some embodiments the tumor contains cells that express a tumor associated antigen selected from the group consisting of f CD19, CD20, CD22, CD30, CD33, BCMA, Igk and ROR1. In some embodiments the tumor contains cells that express the tumor associated antigen CD19.

These and other embodiments of the invention are further described in the “Examples” section of this patent disclosure. All sections of this patent disclosure are intended to be read in conjunction with, and in the context of, all other sections of the present patent disclosure. Furthermore, one of skill in the art will recognize that the various embodiments of the present invention described herein can be combined in various ways, and that such combinations are within the scope of the present invention.

### Examples

CD8+ T cells, critical mediators of adaptive immunity, may also exhibit innate-like properties, such as surface expression of NKG2C, an activating receptor typically associated with Natural Killer cells. We demonstrate that, similar to NK cells, NKG2C⁺TCRαβ⁺CD8⁺ T cells are associated with prior HCMV exposure. Through transcriptomic analysis, we find that the transcription factor BCL11B, a regulator of T cell developmental fate, is significantly down-modulated in NKG2C⁺CD8⁺ T cells when compared to conventional NKG2C⁻CD8⁺ T cells. BCL11B deletion in canonical non NKG2C⁺ CD8 T cells induced expansion of cells with a similar phenotype as seen on naturally occurring NKG2C⁺CD8⁺ T cells, including the surface expression of NKG2C. In addition to expressing several NK cell markers, NKG2C⁺CD8⁺ T cells resemble oligoclonal T_{EMRA} cells and demonstrate the capacity to proliferate without evidence of upregulation of the classic exhaustion marker, PD1. Furthermore, NKG2C⁺CD8⁺ T cells from some individuals exhibit high effector function against leukemia cells and HCMV-infected fibroblasts, dictated by their NKG2C and TCR specificity. Given their intrinsic capacity to recognize diseased cells, NKG2C⁺CD8⁺ T cells represent a novel lymphocyte population that straddles the boundary between innate and adaptive immunity and present an attractive alternative for cellular therapy, including CAR T-based therapies.

Among pathogens, few leave as large an imprint on the immune system as human cytomegalovirus (HCMV)(1), which infects all populations with a 50-100% penetrance(2). In healthy individuals, Natural Killer (NK) and T cells play complementary roles in maintaining HCMV in a latent state. HCMV downregulates classical HLA class I expression on the infected cell, impairing T cell recognition, but potentially increasing its susceptibility to NK surveillance(3).

CMV-specific NK responses have been identified in both mice and humans, and in the former are characterized by increased cytotoxicity and early production of IFN-γ, followed by clonal expansion and recall of virus-specific subsets(4). In humans, the non-classical HLA-E molecule is the ligand for both the activating CD94/NKG2C and the inhibitory CD94/NKG2A heterodimers, which are almost entirely mutually exclusive in their cell surface expression. HLA-E expression is distinctively increased by HCMV(5) through presentation of HCMV-derived UL40 peptides(6), potentially explaining the expansion of NK cells expressing NKG2C(7, 8). NK cells expressing NKG2C are educated by self-HLA-specific inhibitory killer Ig-like receptors (KIR) and expand during primary and secondary infection, but only in some individuals(9). NKG2C expression marks a subset of mature (CD57⁺) NK cells labeled “adaptive NK”, which are deficient in the FcεRIγ chain, the transcription factor PLZF, and the signaling molecules Syk and Eat-2(9). Adaptive NK cells display a heightened response to HCMV, triggered by antibody-dependent cellular cytotoxicity (ADCC), but can still be inhibited through KIR engagement of HLA class I ligands and are hyporesponsive to a broad set of targets(10).

T cell response is critical to resolving and preventing HCMV disease(1). Conventional T cell immunity against HCMV includes cytotoxicity of infected cells, followed by clonal expansion, differentiation, establishment of memory and recall of virus-specific subsets(11). Because of the latent nature of HCMV infection, it is likely that certain HCMV antigens are periodically shed, resulting in lifelong accumulation of virus-specific T cells, with one or a few clones capable of populating up to 30% of the entire T cell memory pool(12).

Over the past two decades, the distinction between innate and adaptive immunity has become blurred. In mice, bacterial and viral pathogens can give rise to innate-like T cells(13). In humans, some individuals harbor TCRαβ CD8 T cells expressing the molecules CD56, ILT2, KIR(14, 15) and NKG2A or NKG2C receptors(16). NKG2C stimulation with HLA-E-expressing targets leads to proliferation and cytotoxicity of NKG2C⁺ T cells, illuminating alternative pathways for T cell activation(16). A clinical benefit to the NKG2C⁺ T cell population has been observed in humans infected with Mycobacterium leprae(17).

Here we show that in HCMV-seropositive individuals, an expanded population of CD8 T cells expresses NKG2C, acquires an NK-like genetic signature and downregulates a set of T cell identity genes, most prominent of which is the transcription factor BCL11B. NKG2C⁺ T cells have a restricted TCR repertoire with preferential use of the VP chain TRBV-14. NKG2C⁺ T cells can be activated by HLA-E engagement and, in those individuals with TRBV-14 TCR restriction, demonstrate high efficiency killing of a set of tumor cells via both TCR and NKG2C engagement. To our knowledge, this is the first evidence in humans of HCMV exposure giving rise to a BCL11BL^{low}NKG2C⁺CD8⁺ T cell population with broad immune surveillance capacity. Their innate ability to respond to infected target cells and tumor cell lines reveals an opportunity for adoptive cellular therapy.

NKG2C⁺ CD8⁺ T Cells are Associated with HCMV Seropositivity in Healthy Donors

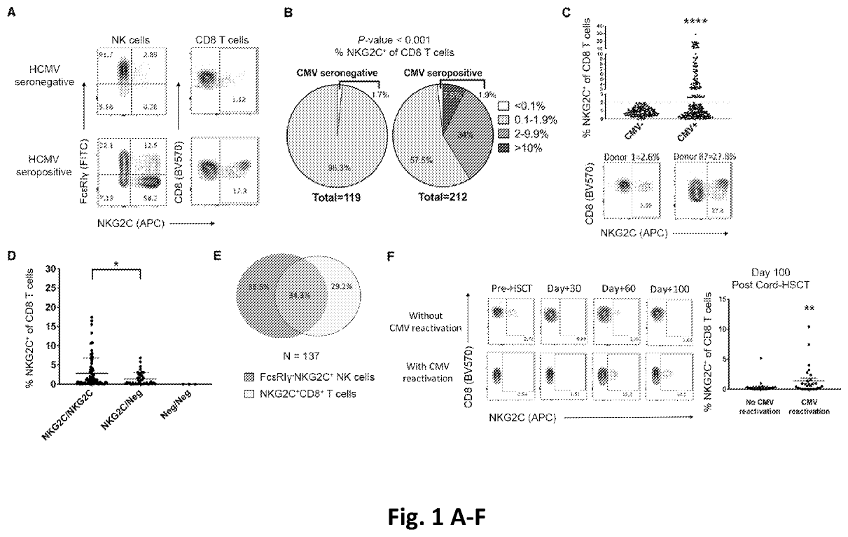

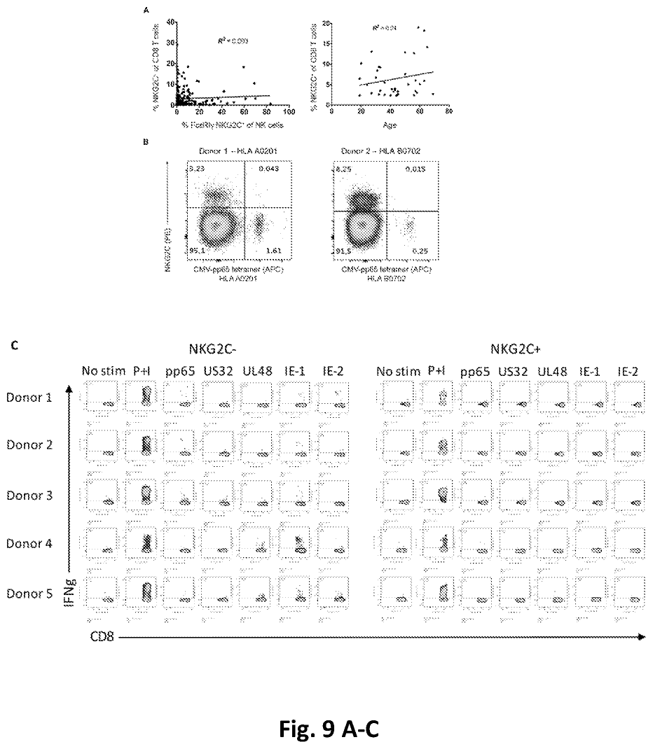

To identify and quantify the human T cell population that expresses NKG2C, we analyzed the peripheral blood from 331 healthy subjects. Of the 331 individuals, 212 (64%) were HCMV-seropositive. In line with previous findings(7, 16), we observed in some individuals that both T cells (TCRαβ) and NK cells express NKG2C. The presence of an “adaptive” NK cell population, phenotypically defined by the lack of FcεRIγ and the expression of NKG2C, was associated with HCMV seropositivity of the donor (FIG. 1A). Likewise, among T cells, NKG2C⁺CD8⁺ T cells were also associated with HCMV-seropositivity. As shown in FIG. 1B, 41.5% of the HCMV-seropositive donors exhibited an expanded population of NKG2C⁺CD8⁺ T cells, as defined by a frequency threshold greater than 2% among total CD8 T cells. It should be noted that all donors (except for three individuals lacking KLRC2, the gene for NKG2C) had a detectable NKG2C⁺CD8⁺ T cell population, but HCMV-seronegative individuals only exhibited a frequency of less than 2% of the total CD8 T cell population. In contrast, in HCMV-seropositive individuals, 41.5% exhibited NKG2C⁺CD8⁺ T cells with frequencies ranging from 2.6% to as much as 27.8% of the total CD8 T cell population (FIG. 1C). The highest frequency was observed in individuals homozygous for the KLRC2 gene (FIG. 1D). Among all HCMV-seropositive donors tested, 137 (65%) had expansion of an NKG2C⁺ population. Among these individuals, 36.5% had expansion in NK cells only, 29.2% in CD8 T cells only and 34.3% in both lymphocyte populations (FIG. 1E). The age of the donor did not influence the magnitude of the population (FIG. 9A). Interestingly, in individuals with an expanded NKG2C⁺CD8⁺ T cell population, NKG2C expression was absent on pp65 CMV-specific CD8 T cells (FIG. 9B). When analyzed for specific reactivity against the top 5 most recognized HCMV open reading frames (ORFs) by CD8⁺ T cells(12), NKG2C⁺CD8⁺ T cell population showed no specific activation (FIG. 9C). Therefore, the presence of a CD8 T cell population expressing NKG2C is independent of the presence of an NKG2C⁺ NK population, expansion of the population is variable and age-independent between individuals, and antigen specificity among NKG2C⁺CD8⁺ T cells is not related to the most common HCMV peptides that dominate canonical CTL response(12, 18, 19).

To provide further evidence of the association between NKG2C⁺CD8⁺ T cells and HCMV infection, we retrospectively examined patients who received umbilical cord blood transplantation. This is a clinical setting with a frequent incidence of HCMV reactivation and permits detection of the emergence and tracking of the expansion of NKG2C⁺CD8⁺ T cells. We identified a clear expansion of NKG2C⁺CD8⁺ T cells in patients with documented HCMV reactivation, but no expansion was observed in patients who did not experience HCMV reactivation (FIG. 1F).

From the serology studies of healthy individuals and from the immune reconstitution data from transplant recipients, we conclude that the expansion of an NKG2C⁺CD8⁺ T cell population is associated with HCMV infection.

NKG2C⁺CD8⁺ T Cells are PD1-Negative and Resemble T_{EMRA} Cells and NK Cells

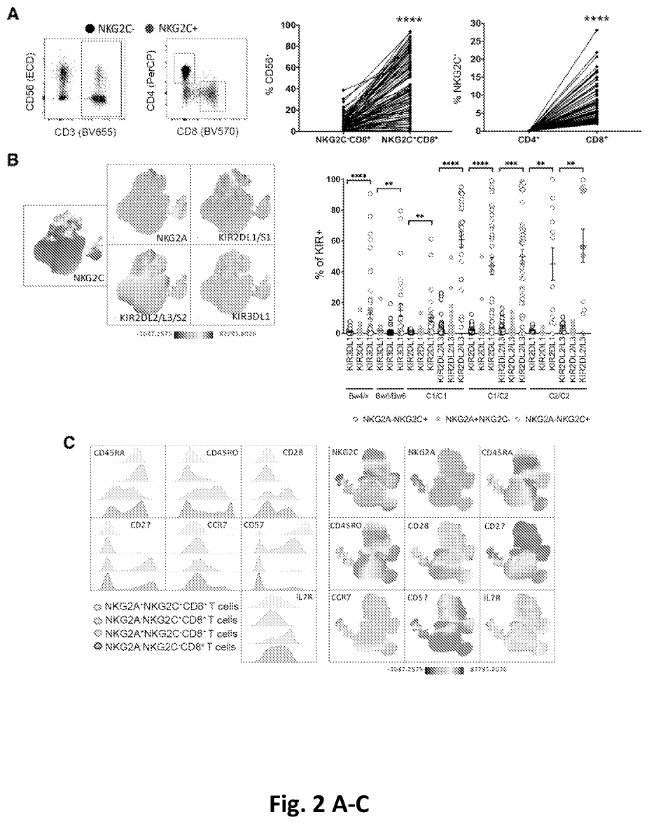

Further phenotyping of NKG2C⁺CD8⁺ T cells revealed that in the majority of donors tested, NKG2C⁺CD8⁺ T cells co-express CD56. We found no NKG2C-expressing cells among CD4 T cells (FIG. 2A). When analyzed for inhibitory receptor surface expression, NKG2C⁺CD8⁺ T cells demonstrate a higher frequency of KIR2DL1/S1-, KIR2DL2/L3/S2- and KIR3DL1-expressing cells compared to their conventional NKG2C⁻CD8⁺ T cell or NKG2A⁺CD8⁺ T cell counterparts, without evidence for skewing of KIR expression in relation to self-HLA class I ligands (FIG. 2B). It is known that CD8 T cells can express the inhibitory CD94/NKG2A receptor and that NKG2A⁺CD8⁺ T cells have decreased capacity for activation and cytotoxicity(20). Interestingly, a minor population of CD8 T cells co-expressing NKG2A and NKG2C could be readily observed (FIG. 2B, UMAP plots).

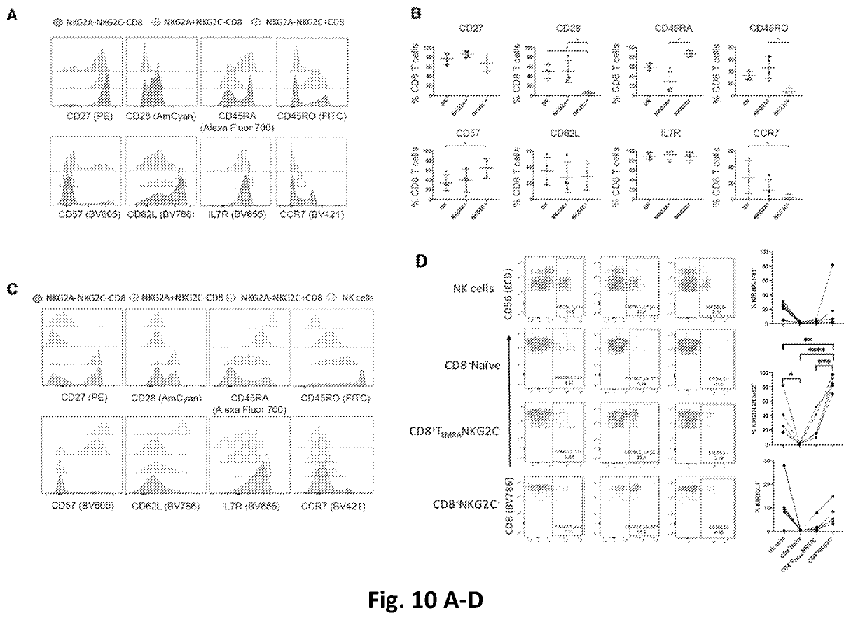

Assessment of T cell differentiation markers revealed that the NKG2C-expressing population is CD45RA⁺CD45RO⁻CD28⁻CD27⁻CCR7⁻CD57⁺IL7^{low/-}, resembling terminally differentiated T_{EMRA} cells. The CD8⁺ population co-expressing both NKG2A and NKG2C exhibited a phenotype similar to the NKG2C⁺ cells (FIG. 2C). In contrast, NKG2A⁺CD8⁺ T cells resembled central memory T cells (CD45RA⁻CD45RO⁺CD28⁺CD27⁺CCR7⁺CD57⁻ IL7R⁺). Interestingly, the small fraction (<2%) of NKG2C+ cells from HCMV-seronegative individuals retained CD27 surface expression, suggesting that the population had not been exposed to antigen-induced activation and expansion (FIG. 10A-B). Of note, the total NK population (predominantly composed by CD56^{Dim} cells) exhibited the same expression pattern as the NKG2C⁺CD8⁺ T cells for the T cell differentiation markers described above, suggesting either that the NKG2C⁺CD8⁺ T population is a true T_{EMRA} population or that it is NK-like (FIG. 10C). When assessed for KIRs surface expression, the frequency of NKG2C⁺CD8⁺ T cells expressing KIR2DL1/S1, KIR2DL2/L3/S2 or KIR3DL1 was much higher than their naïve (CD45RA⁺CCR7⁺) or T_{EMRA} (CD45RA⁺CCR7⁻) counterparts, comparable to those observed on NK cells (FIG. 10D).

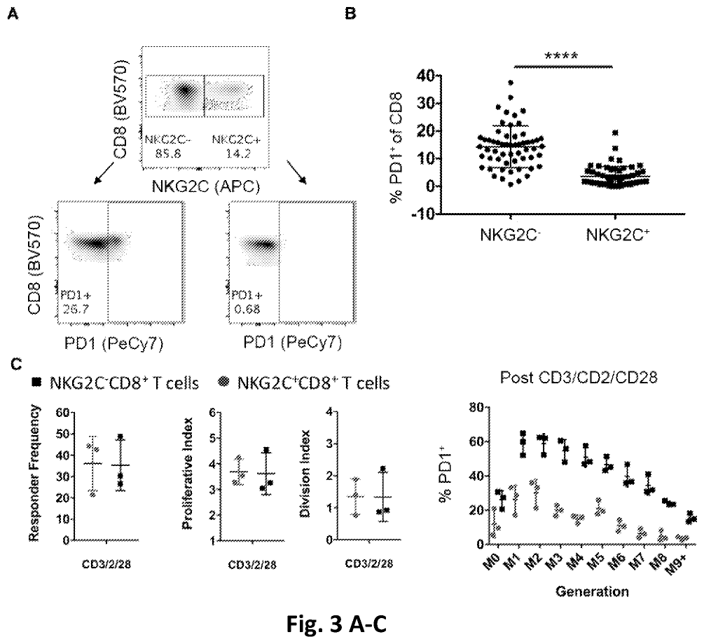

Because classic T_{EMRA} cells are a terminally differentiated population that is more likely than other T cell populations to express exhaustion markers and to have impaired proliferative capacity(21), we investigated if NKG2C⁺CD8⁺ T cells exhibited the same features. We found that the overwhelming majority of NKG2C⁺CD8⁺ T cells are negative for the checkpoint molecule PD-1 at rest, a notable distinction when compared to their NKG2C⁻CD8⁺ T cell counterparts (FIG. 3A-B). Despite differences in surface expression of PD-1, NKG2C⁺CD8⁺ cells display similar proliferation kinetics to NKG2C⁻CD8⁺ T cells after a week of CD3/CD2/CD28 stimulation. Nevertheless, the majority of NKG2C⁺CD8⁺ T cells remained PD-1 negative in every mitotic generation, in contrast to conventional CD8 T cells which upregulated PD-1 expression upon activation. (FIG. 3C).

NKG2C⁺CD8⁺ T Cells Acquire NK Cell Transcriptional Features and Downregulate a BCL11B-Dependent Transcriptional Program.

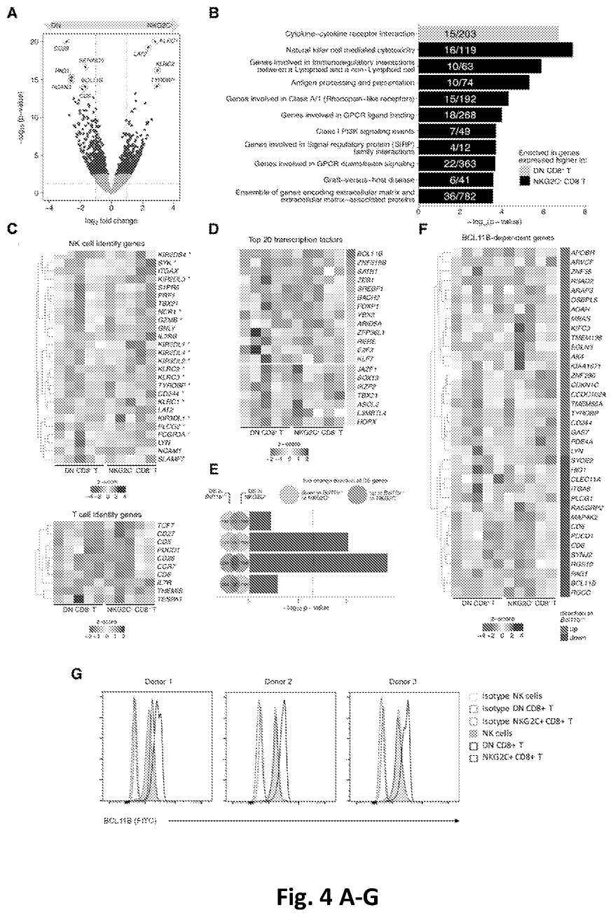

The intriguing mixture of NK-like features within a T cell subset implies a transcriptional signature distinct from conventional CD8 T cells. We performed whole transcriptome analysis on paired samples of NKG2A⁻NKG2C⁺CD8⁺ T cells (NKG2C⁺CD8⁺) and NKG2A⁻NKG2C⁻ CD8⁺ T cells (DN CD8⁺ T) obtained from the peripheral blood of five healthy HCV-seropositive donors. NKG2C⁺ and DN CD8⁺ T cells defined transcriptionally distinct populations (FIG. 4A).

Gene set enrichment analysis revealed that NKG2C⁺CD8⁺ T cells are enriched for transcription of genes associated with the “NK cell-mediated cytotoxicity” pathway, indicating that NKG2C⁺CD8⁺ T cells have a strong NK cell-like signature (FIG. 4B). When the analysis is focused specifically on genes commonly associated with NK or T cell identity, NKG2C⁺CD8⁺ T cells demonstrated higher expression of genes classically associated with NK cells and lower expression genes associated with T cells compared to DN CD8 T cells (FIG. 4C). The finding of such a prominent NK signature enrichment in an unbiased evaluation validates the phenotypic similarities between NK cells and NKG2C⁺CD8⁺ T cells and suggests shared biologic functions.

Among the top 10 differentially expressed (DE) genes between the two populations, was the transcription factor (TF) BCL11B (FIG. 4A). BCL11B was also found to be the highest DE TFs among the top 20 DE TFs (FIG. 4D). NKG2C⁺CD8⁺ T cells in humans exhibited significantly decreased BCL11B transcripts, suggesting that this cell population has lost T cell identity during its development.

Expressed in all T cell subsets and progenitors, BCL11B in mice initiates progenitor cell commitment to the T cell lineage prior to TCR expression and regulates key processes of T cell function and survival(22). BCL11B is considered a master regulator of T cell fate, controlling a transcriptional program that actively prevents development of lymphocytes to an innate NK-like phenotype(23).

To determine if BCL11B loss in human NKG2C⁺CD8⁺ T cells actively regulates a similar transcriptional program, we converted the DE murine genes between BCL11B-knockout and wild-type CD8 T cells(22) to their human orthologs and compared them to the DE genes between NKG2C⁺CD8⁺ and conventional (DN CD8⁺) T cells (FIG. 4E). Using this approach, we find that the pattern of BCL11B-dependent genes, both upregulated and downregulated, is recapitulated in humans when NKG2C⁺CD8⁺ T cell is compared to the conventional CD8 T cell (FIG. 4F). Thus, the induced adoption of an NK phenotype in T lymphocytes following deletion of BCL11B in mice occurs naturally in the human NKG2C⁺CD8⁺ T cell population.

We validated BCL11B protein downregulation by intracellular staining and confirmed that NKG2C⁺CD8⁺ T cells have less BCL11B protein compared to conventional CD8 T cells. Moreover, the low levels of BCL11B protein in NKG2C⁺CD8⁺ T cells are comparable to those observed in NK cells from the same donor (FIG. 4G).

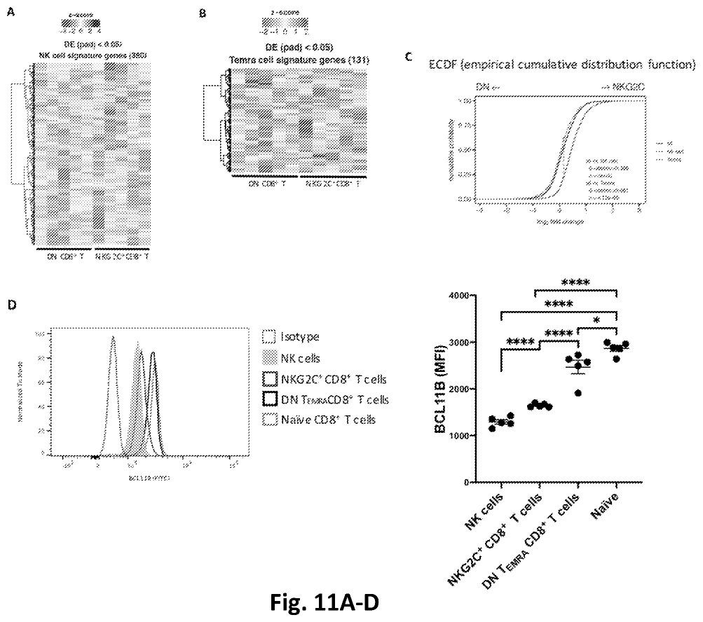

To assess how much of the unique gene signature observed on NKG2C⁺CD8⁺ T cells is to be attributed to their putative T_{EMRA} state or if an “NK reprogramming” is indeed occurring, we first derived an “NK signature” comparing paired NK cell (sorted on CD56⁺ CD3⁻NKG2A⁻ NKG2C⁻) and DN CD8⁺ T cells samples. NKG2C⁺CD8⁺ T cells showed higher expression of genes associated with an “NK signature” compared to the DN CD8⁺ T cells (FIG. 11A). Second, to derive a “T_{EMRA} signature” we took advantage of previously published T_{EMRA} cells transcriptome data (24) and compared it to our newly generated RNA-seq datasets. We didn't observe any clear pattern of expression for DE genes that are part of a T_{EMRA} signature (FIG. 11B). Finally, empirical cumulative distribution function (ECDF) was performed to determine whether the cumulative levels of log₂ fold change in the subset of genes associated with the T_{EMRA} or NK signature was differential compared to all DE genes between NKG2C⁺CD8⁺ and DN CD8⁺ T cells. We observed a general trend for NK cell signature genes to be upregulated in the NKG2C⁺CD8⁺ T cells compared to DN CD8⁺ T cells (FIG. 11C).

Furthermore, NKG2C⁺CD8⁺ T cells showed less BCL11B protein expression than classic NKG2C⁻CD45RA⁺CCR7⁻ T_{EMRA} cells (FIG. 11D).

Taken together, our data show that NKG2C⁺CD8⁺ T cells and NK cells appear phenotypically and transcriptionally closely related, and the dramatic downregulation of BLC11B in NKG2C⁺CD8⁺ T cells and loss of its downstream transcriptional effects likely account for its adoption of an NK-like identity.

BCL11B Deletion Promotes the In Vitro Generation of NKG2C⁺CD8⁺ T Cells.

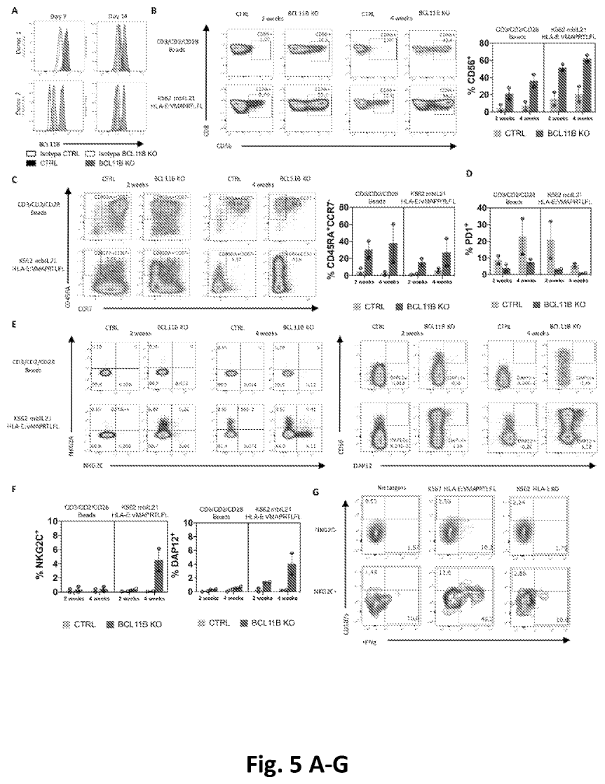

NKG2C⁺CD8⁺ T cells display a pronounced downregulation of BCL11B both at the transcriptional and protein level. To explore the potential role of BCL11B in the generation of the NKG2C⁺CD8⁺ T cells we isolated CD56-NKG2A⁻NKG2C⁻KIRs-CD3⁺ CD8⁺ T cells from two different HCMV-seronegative individuals and nucleofected these cells with CRISPR/Cas-9 RNPs containing sgRNAs targeting BCL11B for knockout (KO) or a control sgRNA (CTRL). Maximum protein loss, as measured by flow cytometry, was achieved after 14 days in culture for both donors (FIG. 5A). Subsequently, both CTRL and KO cells were expanded using either CD3/CD2/CD28 beads or a K562 cell line expressing HLA-E with the HLA-G*01 leader sequence peptide (VMAPRTLFL (SEQ ID NO. 1)), homologous to a high-affinity HCMV UL-40 peptide(6), and membrane bound IL-21 (mbIL-21), a cytokine that enhances resting T cell proliferation in vitro and promotes antigen-specific CD8 T cell expansion in vivo(25). In the absence of BCL11B we observed a dramatic increase in the frequencies of CD56⁺ cells (a surface marker classically associated with NK cells) compared to CTRL cells, under both stimulation conditions (FIG. 5B). Further analysis stratifying the cells for CD45RA and CCR7 expression revealed a preferential expansion of CD45RA⁺CCR7⁻ cells upon BCL11B deletion, not observed in the CTRL cells (FIG. 5C). BCL11B KO cells also failed to upregulate PD1 to the same extent as CTRL cells upon stimulation (FIG. 5D). Strikingly, after 4 weeks of stimulation with HLA-E:VMAPRTLFL (SEQ ID NO. 1) expressing K562 cells, a population of NKG2C⁺ and DAP12⁺ cells emerged upon BCL11B deletion. In contrast, NKG2C⁺ T cell expansion was not observed in CTRL cells or in BCL11B KO cells stimulated with CD3/CD2/CD28 beads (FIG. 5E-F). The BCL11B⁻NKG2C⁺ cells were able to degranulate and produce IFN-γ against HLA-E:VMAPRTLFL (SEQ ID NO. 1) expressing K562 cells, as opposite to BCL11B⁻NKG2C⁻ cells were no activation was observed (FIG. 5G).

Our results provide evidence for a direct role of BCL11B downregulation in promoting an NK-like pathway of T cell differentiation, specifically in inducing a NKG2C⁺CD8⁺ T cell phenotype that expands upon HLA-E ligand stimulation.

NKG2C⁺CD8⁺ T Cells can be Activated Via NKG2C and TCR

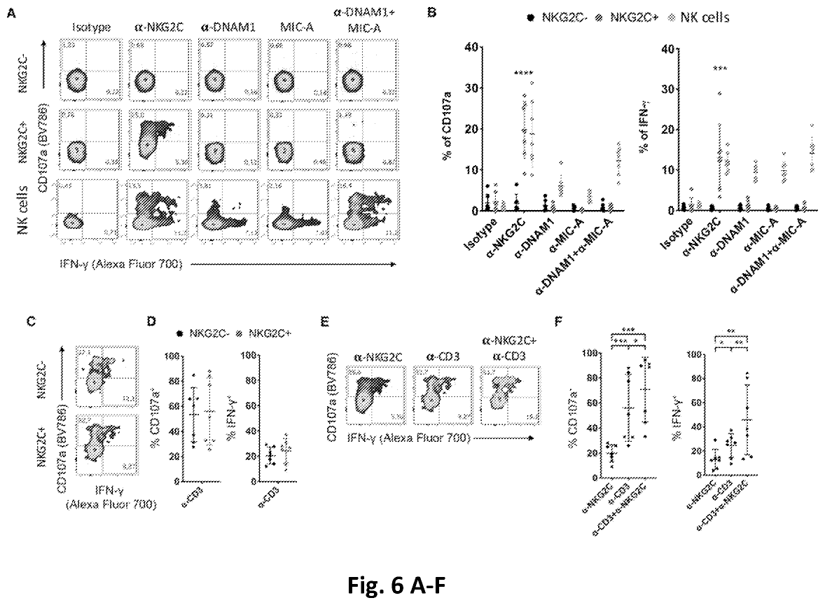

Stimulation of NKG2C by plate-bound antibody increased degranulation and IFN-γ production in NKG2C⁺CD8⁺ T cells, compared to NKG2C⁻CD8⁺ T cells where no significant increase was observed. Antibody triggering of the activating receptors DNAM-1, NKG2D or a combination of both did not induce response (FIG. 6A-B). CD3 stimulation induced high amounts of degranulation and IFN-γ production in both NKG2C⁻ and NKG2C⁺CD8⁺ T cells, indicating that TCR signaling was equally functional in both populations (FIG. 6C-D). Importantly, even when CD3 was stimulated, NKG2C engagement could still increase degranulation and IFN-γ production further in NKG2C⁺CD8⁺ T cells in an additive fashion (FIG. 6E-F).

These results demonstrate that NKG2C stimulation can induce cytotoxic and cytokine effector functions, highlighting an alternative non antigen-restricted pathway for activation of this T cell population, in addition to their intact TCR signalling. Furthermore, co-triggering of NKG2C and CD3 enhances CD8 T cell response beyond each individually, thereby diversifying the responsiveness and broadening the activation threshold of the cell.

NKG2C⁺CD8⁺ T Cells Display Potent Anti-Tumor and Anti-HCMV Effector Functions Mediated by their NKG2C and TCR Specificity.

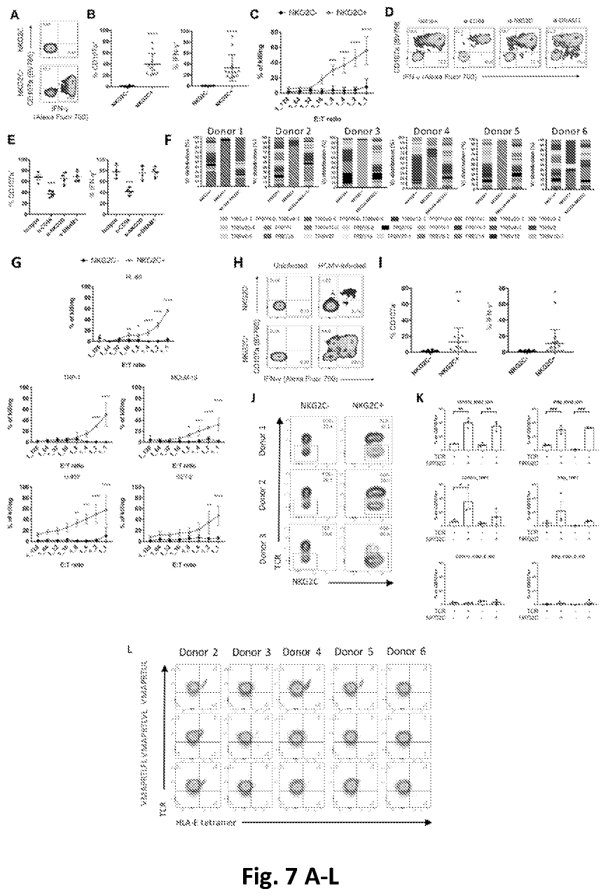

It has been reported that patients chronically infected with Mycobacterium leprae who harbor a NKG2C⁺CD8⁺ T cell population have improved outcomes(17). To determine if NKG2C⁺CD8⁺ T cells respond to targets through NKG2C, we co-incubated them with the K562 cell line expressing HLA-E with the HLA-G*01 leader sequence peptide (VMAPRTLFL (SEQ ID NO. 1)). NKG2C⁺CD8⁺ T cells robustly responded, as measured by CD107a mobilization and IFN-γ production, whereas the NKG2C⁻CD8⁺ T cells did not (FIG. 7A-B). NKG2C⁺CD8⁺ T cells effectively killed the HLA-E:VMAPRTLFL (SEQ ID NO. 1) expressing targets, whereas conventional CD8 T cells did not (FIG. 7C). Blocking NKG2C with an antibody against CD94, we observed a significant reduction in CD107a mobilization and IFN-γ production against the K562 HLA-E:VMAPRTLFL (SEQ ID NO. 1) target cell line, indicating contribution from the receptor (FIG. 7D-E). In contrast, blocking NKG2D or DNAM-1 receptors, highly expressed on NKG2C⁻CD8⁺ T cells (data not shown), did not have any effect. Complete blockade of NKG2C⁺CD8⁺ T cell functionality could not be achieved, however, suggesting that a separate receptor, possibly the TCR, contributed to target cell recognition.

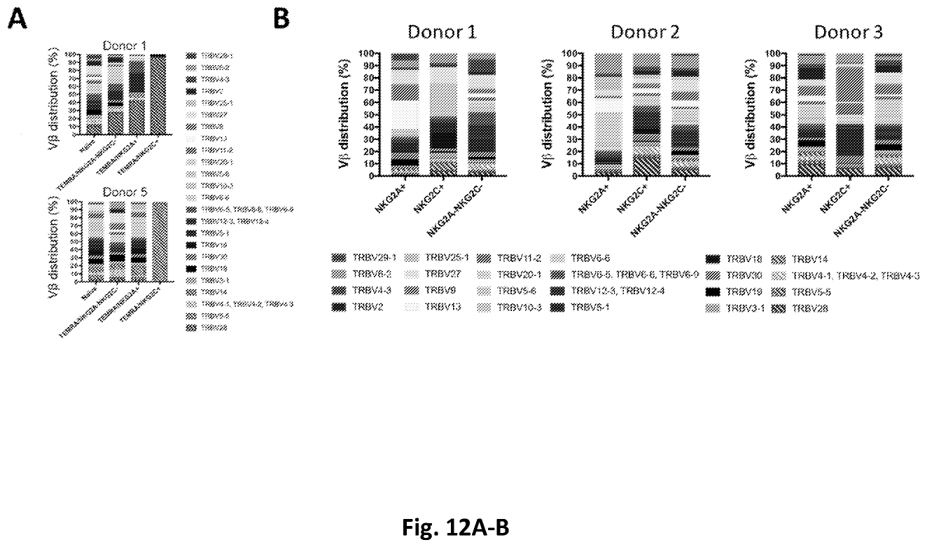

In light of these results, we first analyzed in further details TCR Vβ usage by NKG2C⁺CD8⁺ T cells in six individuals using a panel of 24 TCR Vβ antibodies to stain NKG2A⁺NKG2C⁻, NKG2A⁻NKG2C⁺ and NKG2A⁻NKG2C⁻ CD8⁺ subpopulations. In all individuals, the majority of the NKG2C⁺CD8⁺ T cells expressed one prominent TCR Vβ chain, to near complete domination of the population in four of the six individuals. In contrast, all other subpopulations, including those expressing NKG2A, demonstrated a diversity of TCR Vβ usage (FIG. 7F). Interestingly, in 50% of individuals, we observed a skewed distribution towards a single dominant Vβ chain: TRBV-14. To determine if other CD45RA⁺CCR7⁻ populations, including canonical T_{EMRA} cells, demonstrate a similarly narrow TCR Vβ chain repertoire, we analyzed in 2 different individuals TCR Vβ chain usage, stratifying the CD8 T cell population into canonical T_{EMRA} (CD45RA⁺CCR7⁻) and the T_{EMRA}-like NKG2C⁺CD8⁺ population, comparing both to naïve T cells (CD45RA⁺CCR7⁺). As expected, the NKG2C⁺CD8⁺ population exhibited the narrowest TCR Vβ chain repertoire (FIG. 12A). A small population of NKG2A⁺ CD45RA⁺CCR7⁻ cells could also be identified in most individuals and, like conventional T_{EMRA} cells, demonstrated polyclonal TCR Vβ chain usage. NKG2C⁺CD8⁺ T cells from HCMV seronegative donors exhibited a polyclonal repertoire, comparable to that observed in NKG2C⁻CD8⁺ cells from the same individuals (FIG. 12B).

The sequence of CDR3 and the identity of the flanking V and J gene segments are widely used to classify TCR variants(26). To identify unique CDR3 regions, we took advantage of iPair next-generation sequencing (NGS) technology to specifically amplify the productive α and β TCR transcripts across the CDR3 in the NKG2C⁺CD8⁺ T cells of three donors previously analyzed by flow cytometry for TCR Vβ usage. Sequencing of NKG2C⁺CD8⁺ T cell clones confirmed the predominance of single Vβ family usage within each donor (Table 1). Of the 21 productive CDR3 sequences identified in Donor 1, 19 (86%) showed TRBV-28 usage. For donors 4 and 5, we also observed predominance of one Vβ use, with 26 of 29 clones (90%) in donor 4 and 21 of 22 clones (95%) in donor 5 exhibiting TRBV-14 usage. In all three donors, the majority of clones exhibited identical CDR3 sequences, suggestive of clonal expansion (data not shown).

TABLE 1
Single-cell sequencing of the productive α and β
TCR pairs expressed by NKG2C+CD8+ T cells

| Donor | β_V Gene | α_V Gene | Clones |
|---|---|---|---|
| 1 | hTRBV19 | hTRAV21 | 1 |
| 1 | hTRBV28 | hTRAV21 | 18 |
| 1 | hTRBV28 | hTRAV25 | 1 |
| 1 | hTRBV5-1 | hTRAV30 | 1 |
| 4 | hTRBV12-3 | hTRAV5 | 1 |
| 4 | hTRBV12-4 | hTRAV9-2 | 2 |
| 4 | hTRBV14 | hTRAV13-1 | 3 |
| 4 | hTRBV14 | hTRAV38-1 | 15 |
| 4 | hTRBV14 | hTRAV8-3 | 4 |
| 4 | hTRBV14 | hTRAV12-1 | 4 |
| 5 | hTRBV10-2 | hTRAV38-2/DV8 | 1 |
| 5 | hTRBV14 | hTRAV29/DV5 | 21 |

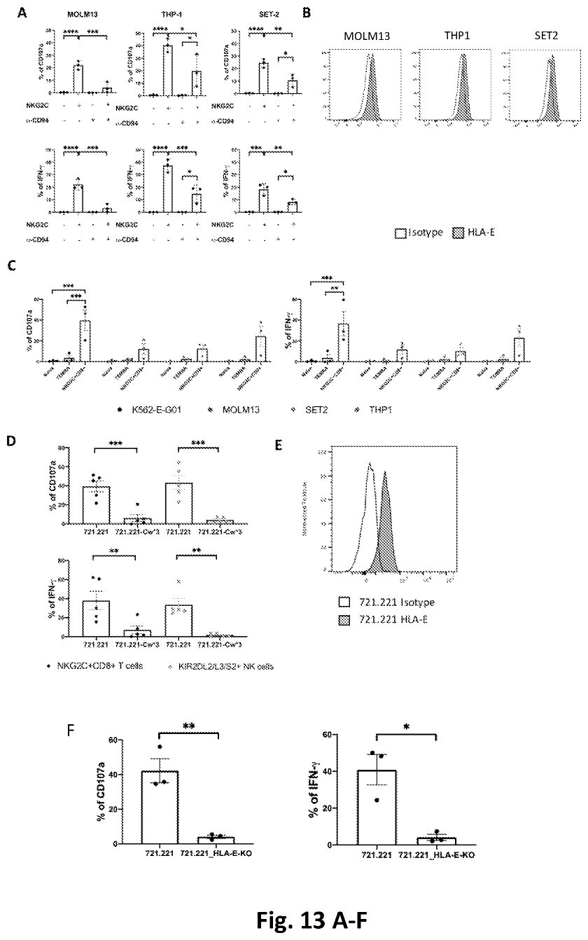

Given the difference in TCR usage across donors, we investigated if that variability could influence the recognition of a series of allogeneic acute myeloid leukemia (AML) cell lines. While NKG2C⁺CD8⁺ T cells from all donors tested were able to recognize HLA-E:VMAPRTLFL (SEQ ID NO. 1) expressing K562 (FIG. 7A-B), only those exhibiting a TRBV-14 usage demonstrated significantly higher killing capacity against all six leukemia cell lines than their NKG2C⁻CD8⁺ T cell counterpart (FIG. 7G). CD94 blockade reduced CD107a mobilization and IFN-γ production by NKG2C⁺CD8⁺ T cells against the AML targets (FIG. 13A) but again, complete blockade of NKG2C⁺CD8⁺ T cell functionality could not be reached. The AML targets all display HLA-E surface expression (FIG. 13B).

When total NKG2C⁻CD8⁺ T cells were stratified according to their T_{EMRA} (CD45RA⁺CCR7⁻) or naïve (CD45RA⁺CCR7⁺) phenotype the NKG2C⁺CD8⁺ T cells showed the highest CD107a mobilization and IFN-γ production compared to the other subsets (FIG. 13C).

We then assessed activation by NKG2C⁺CD8⁺ T cells against biopsy-derived human fibroblasts (HFs) infected with the TB-40E HCMV strain(27). NKG2C⁺CD8⁺ T cells displayed a strong anti-HCMV reactivity, compared to NKG2C⁻CD8⁺ T cells from the same individuals (FIG. 7H-I), and were not reactive to uninfected cells.

We next investigated if the TCR was involved in the recognition of the allogeneic AML target cells. We performed a TCR knockout using CRISPR/Cas-9 RNPs targeting the TRAC locus from the three broadly reactive donors having a NKG2C⁺CD8⁺ T cell population. After 4 days in culture a proportion of both NKG2C⁻ and NKG2C⁺ cells showed no surface TCRαβ expression, as measured by flow cytometry (FIG. 7J). We did not observe any significant reduction in CD107a mobilization and IFN-γ production by NKG2C⁺TCRαβ⁻CD8⁺ T against the K562 HLA-E:VMAPRTLFL (SEQ ID NO. 1) target cell line, indicating that the NKG2C receptor on CD8⁺ T cells can be potently activated by the HLA-G*01 leader sequence peptide VMAPRTLFL (SEQ ID NO. 1), as previously seen for NKG2C⁺ NK cells(6), and that the TCR is not specific for this UL-40 derived HCMV sequence (FIG. 7K, upper panel). On the contrary, the reactivity of NKG2C⁺CD8⁺ T cells against the AML cell line THP-1 was partially abrogated in the absence of TCRαβ expression, confirming contribution of the TCR receptor (FIG. 7K, middle panel). The absence of HLA-E expression on the target cells completely abrogated the functional response of the NKG2C⁺CD8⁺ T cells (FIG. 7K, lower panel), indicating that the TCR is restricted to epitopes presented by HLA-E.

To further support data on the specificity of the TCR of NKG2C⁺CD8⁺ T cells for HLA-E-bound peptides, HLA-E*01:03 tetramers folded with VMAPRTLIL (SEQ ID NO. 2), VMAPRTLVL (SEQ ID NO. 3) or VMAPRTLFL (SEQ ID NO. 1) were used to stain the CD8⁺ T cells from 5 different donors, in the presence of a α-CD94 mAb to avoid interference of the NKG2A/NKG2C receptors with binding of the HLA-E tetramer to the TCR. The VMAPRTLIL (SEQ ID NO. 2), VMAPRTLVL (SEQ ID NO. 3), and VMAPRTLFL (SEQ ID NO. 1) peptides are present in the leader sequence of the UL40 protein derived from different CMV strains but they are also part of the leader sequence of various HLA class I alleles(28). The majority of TCR⁺ cells from donor 2, 4 and 5 showed binding to the HLA-E tetramer folded with the VMAPRTLIL (SEQ ID NO. 2) sequence, while donor 3 and 5 did not show any significant tetramer binding (FIG. 7L). Donor 2, 4 and 5 were the same donors that displayed a dominant TRBV-14 Vβ chain usage and had a broad functional ability in recognizing several AML targets. When the HLA class I genotype of the donors and targets cells was analyzed (Table 2) we observed that for donors 2, 4 and 5, VMAPRTLIL (SEQ ID NO. 2) is a non-self peptide because it is not present in their HLA-Cw alleles. On the other hand, donors who express HLA-Cw alleles carrying the VMAPRTLIL (SEQ ID NO. 2) peptide (donors 3 and 6) fail to generate CD8⁺ T cells specific for this self peptide (FIG. 7L) and did not functionally react against the allogeneic AML targets (data not shown).

TABLE 2
HLA class I genotypes of effector and target cells

| | | HLA-A | HLA-A | HLA-B | HLA-B | HLA-C | HLA-C |
|---|---|---|---|---|---|---|---|
| Effectors | Donor 1 | A*02:05 | A*24:02 | B*35:02 | B*39:01 | C*04:01 | C*12:03 |
| | Donor 2 | A*02:01 | A*23:01 | B*18:01 | B*49:01 | C*07:01 | C*07:01 |
| | Donor 3 | A*01:01 | A*02:01 | B*07:05 | B*58:01 | C*07:01 | C*08:02 |
| | Donor 4 | A*24:02 | A*26:01 | B*15:35 | B*35:01 | C*07:01 | C*07:01 |
| | Donor 5 | A*01:01 | A*66:01 | B*08:01 | B*41:02 | C*07:01 | C*17:01 |
| | Donor 6 | A*02:01 | A*24:02 | B*07:02 | B*15:01 | C*03:03 | C*07:0 |
| Tumor cell lines | U937 | A*03:01 | A*03:01 | B*51:01 | B*18:01 | C*07:01 | C*01:02 |
| | HL60 | A*01:01 | A*01:01 | B*57:01 | B*57:01 | C*06:02 | C*06:02 |
| | MOLM13 | A*31:01 | A*26:01 | B*51:01 | B*44:03 | C*14:03 | C*14:02 |
| | SET2 | A*33:03 | A*02:01 | B*44:03 | B*40:02 | C*14:03 | C*03:03 |
| | THP1 | A*02:01 | A*02:01 | B*15:11 | B*15:11 | C*03:03 | C*03:03 |
| | SET2 | A*33:03 | A*02:01 | B*44:03 | B*40:02 | C*14:03 | C*03:03 |
| | NALM6 | A*01:01 | A*02:01 | B*08:01 | B*15:01 | C*04:01 | C*07:01 |
| Human fibroblasts | HF1 | A*02:01 | A*03:01 | B*07:02 | B*07:02 | C*02:02 | C*07:02 |
| | HF2 | A*02:01 | A*24:02 | B*15:01 | B*40:02 | C*02:02 | C*04:01 |
| | HF3 | A*01:01 | A*02:01 | B*07:02 | B*40:01 | C*05:01 | C*07:02 |
| | HF5 | A*01:01 | A*02:01 | B*08:01 | B*27:05 | C*02:02 | C*07:01 |

We have previously shown that the NKG2C⁺CD8⁺ T cell population expresses KIRs (FIG. 2B). To investigate if functional inhibition could be mediated by KIR2DL receptors, we incubated KIR2DL2/L3⁺NKG2C⁺CD8⁺ T cells with 721.221 target cells transfected with the HLA-C1 allele Cw*03. The presence of KIR2DL2/L3 cognate ligand HLA-Cw*03 abolished CD107a mobilization and IFN-γ production by NKG2C⁺CD8⁺ T cells, similar to what observed for NK cells (FIG. 13D), indicating that KIR receptors are fully functional. Of note, the NKG2C⁺CD8⁺ T cells showed functional response against wild type 721.221 cells, a HLA A-B-C-deficient cell line, mediated by HLA-E surface expression (FIG. 13E-F).

Overall, TCR studies of the NKG2C⁺CD8⁺ T cell population establish that in some HCMV-seropositive individuals, the NK-like T cells undergo significant clonal expansion in vivo, often bearing a TRBV-14-derived TCR targeting a non-self HLA-E-presented peptide. Combined with broader recognition of HLA-E through NKG2C, NKG2C⁺CD8⁺ T cells from these individuals display greater antitumor, anti-viral reactivity and cytotoxic potential compared to conventional CD8 T cells.

NKG2C⁺CD8⁺ T Cells Proliferate in Response to HLA-E and Represent a Novel Platform for CAR-T Cell Manipulation.

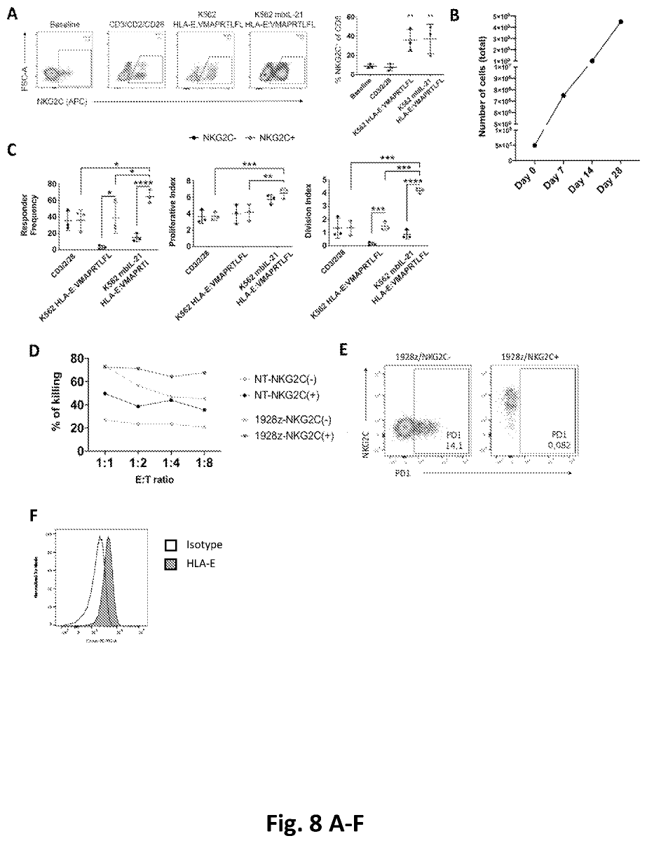

We examined if HLA-E can induce proliferation of activated NKG2C⁺CD8⁺ T cells by co-incubating peripheral blood mononuclear cells (PBMC) with the K562 cell line expressing HLA-E:VMAPRTLFL (SEQ ID NO. 1). Following one week of stimulation, an increase in the frequency of total NKG2C⁺CD8⁺ T cells could be observed, indicating expansion of the cell population (FIG. 8A). Co-incubation of NKG2C⁺CD8⁺ T cells with the K562 cell line expressing HLA-E:VMAPRTLFL (SEQ ID NO. 1) and membrane bound IL-21 (mbIL-21), also induced expansion of the NKG2C⁺CD8⁺ T cell population (FIG. 8A), up to a 1000-fold increase after 28 days of stimulation (FIG. 8B)

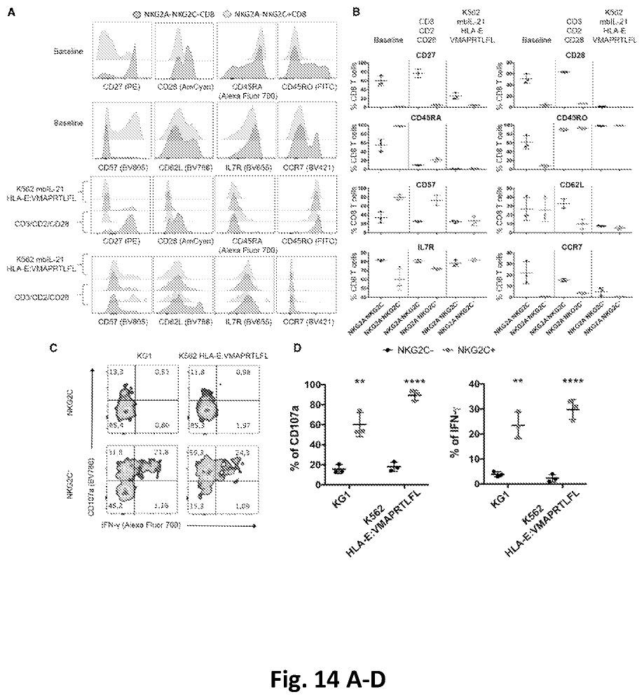

Responder frequency and division index were also higher among NKG2C⁺CD8⁺ T cells in response to both K562 HLA-E:VMAPRTLFL (SEQ ID NO. 1) and K562 HLA-E:VMAPRTLFL (SEQ ID NO. 1) mbIL-21, compared to NKG2C⁻CD8⁺ T cells, whereas no difference was observed between the two populations following CD3/CD2/CD28 stimulation (FIG. 8C). After a 7-day stimulation with either CD3/CD2/CD28 beads or K562 HLA-E:VMAPRTLFL (SEQ ID NO. 1) mbIL-21, NKG2C⁺CD8⁺ T cells acquired surface expression of CD45RO (FIG. 14A-B) and maintained higher capacity to degranulate and produce IFN-γ compared to the NKG2C⁻CD8⁺ T cell counterpart (FIG. 14C-D). Taken together, our data demonstrate that HLA-E on the target cell can provide an extrinsic signal for selective proliferation.

Having demonstrated the rapid and extensive expansion upon HLA-E stimulation, NKG2C⁺CD8⁺ and NKG2C⁻CD8⁺ T cells from one donor were isolated and, after two weeks of expansion using K562 HLA-E:VMAPRTLFL (SEQ ID NO. 1) mbIL-21 they were transduced with the CD19-specific CAR construct 1928z. When co-cultured with the CD19-expressing targets NALM-6 the 1928z-NKG2C⁺CD8⁺ cells demonstrated higher killing capacity than their 1928z-NKG2C⁻CD8⁺ T cell counterpart, especially at low E:T ratios (FIG. 8D). Contrary to NKG2C⁻CD8⁺ T cells, the NKG2C⁺CD8⁺ cells did not show any PD-1 surface expression (FIG. 8E). Basal NALM-6 recognition by NT-NKG2C⁺CD8⁺ T cells was also higher compared to NT-NKG2C⁻CD8⁺ T cells. Notably, NALM-6 express high levels of HLA-E on the surface (FIG. 8F).

These results illustrate how the dual recognition capacity of these cells through NKG2C and the native TCR can be leveraged by introducing CAR expression or, potentially, replacing the native TCR for one that recognizes cancer-specific antigens, while maintaining broad recognition of cellular abnormality via NKG2C:HLA-E and resistance to exhaustion.

The classic concept of the existence of a phenotypic and functional distinction between the innate and adaptive arms of immunity and their respective lymphocyte populations has recently been challenged.

A recent study demonstrated that CD8 T cells can signal through NKp30, another protein classically associated with NK cells(29). Prior studies identified the presence of a CD8 T cell subset that expresses the CD94/NKG2C heterodimer(30, 31) and suggested that the NKG2C may constitute an alternative T-cell activation pathway capable of driving the expansion of and triggering the effector functions of a CTL subset(16).

The origin of the NKG2C⁺CD8⁺ T cell population, the factors leading to its expansion, and the functional peculiarities of this lymphocyte population, however, have been unexplored.

Here we describe a population of CD8 T cells expressing NKG2C in the peripheral blood of healthy HCMV-seropositive individuals. These “unconventional” T cells, although already committed to the CD8-single positive stage of T cell development, have lost expression of the transcription factor BCL11B and have become phenotypically and functionally similar to NK cells. While NKG2C⁺CD8⁺ T cells may be present in the peripheral blood of healthy HCMV-seronegative individuals, their frequency notably remains below 2%. Together with the observation that the population expands in the setting of HCMV reactivation following allogeneic hematopoietic cell transplantation, these findings indicate strongly that HCMV infection is the primary stimulus leading to the expansion of the innate-like NKG2C⁺CD8⁺ T cells.

Despite the likelihood that both NKG2C⁺ lymphocytes expand to HCMV infection, there is no apparent correlation between expansion of NKG2C⁺ NK cells and expansion of NKG2C⁺CD8⁺ T cells in HCMV-seropositive individuals. Furthermore, it is not clear why some individuals exclusively develop NKG2C⁺ NK cells or NKG2C⁺CD8⁺ T cells, or why some expand both populations or neither. However, NKG2C⁺CD8⁺ T cells were observed at a higher frequency on average in HCMV-seropositive individuals homozygous for the NKG2C gene KLRC2. A similar impact of KLRC2 copy number on NKG2C frequency was previously described for NK cells(32).

The T cell response to HCMV is known to be robust and directed against many peptides of the virus(33). Prior studies have primarily focused on pp65-specific T cells, but it is possible that HCMV-specific CD8 T cells reactive toward epitopes generated from other viral proteins could have different phenotypes. Selective expansion may be related to HCMV viral strain and viral peptide presentation by HLA-E(6), which also presents leader peptides of HLA class I molecules(34). Viral strain variation has been proposed as a reason for expansion of the NKG2C⁺ NK cells in some individuals; however, this alone would not explain why only one lymphocyte population (NK vs T cells) would expand and not the other in the same individual, when they both express the same receptor. Specificity of the co-expressed TCR on the NKG2C⁺CD8⁺ T cells likely plays a role, a hypothesis supported by the observation that the expanded population of NKG2C⁺CD8⁺ T cells is highly oligoclonal. Together with the finding of a polyclonal nature observed on the tiny fraction of NKG2C⁺CD8⁺ T cells found in HCMV seronegative individuals, these results support the concept of a strong antigen-driven selection happening in vivo in HCMV seropositive subjects that sustains the expansion of NKG2C⁻ expressing clones.

Expression of inhibitory KIR2DL1, KIR2DL2/L3 and KIR3DL1 were observed in the NKG2C⁺CD8⁺ T cells. This observation is similar to previous reports, where expression of these KIRs was found on terminally differentiated (CD45RA⁺ CD57⁺CCR7⁻CD27⁻CD28⁻IL7R⁻) CD8 T cells(15, 35). Unlike NKG2C⁺ adaptive NK cells, which preferentially express self-HLA-specific inhibitory KIR, self- and nonself-specific KIR appear to be randomly distributed on CD8 T cells, without any clear contribution to the response capacity of the cell (15). In our study and in line with previous observations on other T cell subsets(36), the CD8 T cell population expressing KIRs and NKG2C can be functionally inhibited by cognate HLA class-I molecules, indicating that a self-regulating mechanism to control TCR responses is taking place possibly to avoid cross-reactivity against self-antigens.

Ex vivo phenotype analysis reveals that NKG2C⁺CD8⁺ T cells resemble the T_{EMRA} subset, a classification of memory CD8 T cells associated with chronic infections, including HCMV(37). We observed, however, that NK cells show the same expression pattern for the markers commonly used to classify T_{EMRA} cells, indicating that this phenotype could be associated with the overall T to NK cell shift rather than a terminally differentiated T cell status.

Indeed, after prolonged stimulation, NKG2C⁺CD8⁺ T cells can reacquire expression of CD45RO. Furthermore, the transcriptional profile of classic T_{EMRA} cells appears to be distinct from that observed on NKG2C⁺CD8⁺ T cells, which seem to be more closely related to NK cells.

It is known that during chronic viral infections and in the setting of malignancy, cellular immunity may be impaired due to the upregulation of cell surface exhaustion molecules, such as PD-1. In our study, absence of PD-1 expression is an exceptional feature of the NKG2C⁺CD8⁺ T cell, suggesting a TCR with low affinity and avidity, as has been reported for melanoma-specific CD8 T cells with low PD-1 expression(38, 39). This characteristic of NKG2C⁺CD8⁺ T cells could be critical to ensure long-term proliferation and survival of the cells(40).

Through examination of the gene expression signature of the NKG2C⁺CD8⁺ T cell population, we unexpectedly found that the transcription factor BCL11B is markedly downregulated. A zinc-finger transcription factor required for the development of T cells in mice(41), BCL11B is first expressed at the CD4⁻CD8⁻ stage of T cell development, and germline deletion of BCL11B leads to T cell decreased sensitivity to Notch signaling and differentiation into NK cells(42). In this study, we find that BCL11B downregulation occurs naturally in a T cell subpopulation and appears to be a marker for a general NK reprogramming of a mature T cell, as supported by the transcriptional upregulation of genes classically associated with “NK cell identity” and “NK cell cytotoxicity” and the simultaneous downregulation of genes associated with “T cell identity”. Little has been known about the transcription factors that control the NK program in NKG2C⁺CD8⁺ T cells. Transcriptome comparison with a murine dataset where BCL11B was deleted in young adult thymocytes at the DN2 stage of T cell development(22) reveals that the majority of genes observed to be differentially expressed in naturally occurring human NKG2C⁺CD8⁺ T cells are modulated in the same direction in the BCL11B-knockout murine cells. Strikingly, BCL11B deletion in canonical mature NKG2C⁻CD8⁺ T cells gave rise to CD45RA⁺CCR7⁻CD56⁺NKG2C⁺DAP12⁺ cells, providing evidence to the possibility that lack of BCL11B is responsible for the overall reprogramming of NKG2C⁺CD8⁺ T cells into NK-like cells.

Of note NKG2A⁺ CD8⁺ T cells were also able to expand upon BCL11B deletion. This represents a paradox, given the fact that stimulation with HLA-E expressing targets should suppress the expansion of NKG2A⁺ cells for which HLA-E represents an inhibitory signal. It has been suggested that NK cells do not downregulate the expression of CD94/NKG2A receptors upon ligation(43). NKG2A appear to be constantly recycled at a very slow rate and HLA-E can serially trigger its expression to maintain the inhibitory signal(44). A similar mechanism of sustained NKG2A surface expression can take place in T cells, as supported by our data.

Absence of BCL11B could also represent a putative mechanism for the resistance to PD1 upregulation observed in NKG2C⁺CD8⁺ T cells. In our transcriptome analysis PDCD1 was found to be controlled by BCL11B both in the human and murine dataset. Secondary, NK cells (which express BCL11B at similar low levels than NKG2C⁺CD8⁺ T cells) have also been reported to have minimal to no PD1 expression(45), further supporting a role for BCL11B (and the transcriptional program controlled by it) in regulating the similarities between the two lymphocyte subsets.

TCR analysis indicates that these reprogrammed T cells have undergone significant clonal expansion in HCMV-seropositive individuals, suggesting that a strong response to a pathogenic challenge occurs in vivo. Whether reprogramming of the CD8 T cell occurs after TCR engagement and expansion of the T cell clone or it is an intrinsic characteristic of a T cell subset that during development is diverted from a “conventional” CD8 T cell program, is unclear. As our data suggest, the presence of a small fraction of CD8 T cells displaying NKG2C surface expression in HCMV-seronegative individuals would point towards the second hypothesis.

The finding that NKG2C activation of CD8 T cells can trigger cytotoxic granule release and IFN-γ production in the absence of CD3 stimulation reveals a TCR-independent and innate-like pathway for activation of this population. Moreover, NKG2C signaling complements TCR signaling in activation of the T cell, revealing two non-overlapping pathways that serve to enhance each other. NKG2C⁺CD8⁺ T cells derived from multiple individuals were able to mount a robust and efficient cytotoxic response against HLA-E:VMAPRTLFL (SEQ ID NO. 1)-expressing targets and HCMV-infected targets, which are known to upregulate HLA-E upon HCMV infection(46). The HLA-G-derived leader sequence VMAPRTLFL (SEQ ID NO. 1) represents an optimal ligand for CD94:NKG2C receptors(47, 48). We found that the TRBV-14 TCR expressed by NKG2C⁺CD8⁺ T cells is not specific for the self-peptide VMAPRTLFL (SEQ ID NO. 1), but rather a non-self HLA-E-presented peptide. Thus, these cells exhibit an HLA-E-restricted TCR that cooperates with NKG2C for target cell recognition. Although HLA-E expression on healthy tissue is relatively low compared to classical HLA-class I molecules, it is frequently overexpressed in many types of tumors(49). NKG2C⁺CD8⁺ T cells could represent in the tumor context an effector population with potent immunosurveillance capabilities through its interaction with HLA:E molecules.

Previous reports have elucidated a clinical benefit to a population of polycytotoxic T cells expressing NKG2C in leprosy and tuberculosis(17, 50), suggesting that NKG2C could be used as a biomarker to monitor protective CD8 T cell responses to intracellular pathogens. Beyond this, however, the innate capacity of the NKG2C⁺CD8⁺ T cell population to respond to virally infected cells and tumor cells, combined with their capacity for ex vivo proliferation and resistance to PD-1 upregulation, reveal a potential utility as a cellular immunotherapy, either unmodified or as a cellular vehicle for modification with a chimeric antigen receptor. As supported by our data, 1928z⁺NKG2C⁺CD8⁺ T cells were superior effector cells compared to non NKG2C-expressing cells from the same donor.

Finally, our findings have important implications for the development and differentiation of T and NK cell lineages. Downregulation of BCL11B transcription in NKG2C⁺CD8⁺ T cells indicates that a lymphocyte already committed to the T cell lineage can still be diverted toward an innate lymphoid fate, gaining innate capacity to respond through NKG2C without sacrificing any ability to signal via the native TCR. What molecular events trigger this diversion late in T cell development remains unknown but raises the possibility of reprogramming T cells to adopt NK-like features for immune advantage by targeting BCL11B.

### Materials and Methods

### Study Design

This study was aimed at characterizing the human CD8⁺ T cell subset that expresses NKG2C. Peripheral blood samples were collected from healthy human donors. DNA was extracted from whole blood and KLRC2 zygosity was determined by PCR(51) for 130 individuals. Researchers were not blinded as to the source of the blood samples and HCMV serostatus.

### Cell Sources and Preparation

Peripheral blood mononuclear cells (PBMC) were isolated by Ficoll (GE Healthcare) centrifugation. Additional PBMC were isolated from buffy coats obtained from healthy volunteer donors. PBMC were cryopreserved in fetal bovine serum with 10% DMSO. Human cytomegalovirus (HCMV) serostatus was determined. A total of 59 adult patients who underwent Double Umbilical Cord Blood (DUCB) hematopoietic cell transplants (HCT) between 2006 and 2017 were included in the study, n=30 who did not experience HCMV reactivation and n=29 who did experience HCMV reactivation. Patients provided informed written consent for research, and studies were approved by an institutional review board.

### Flow Cytometry

PBMC (2×10⁵ cells per well) were stained for 20 minutes with LIVE/DEAD™ Fixable Aqua—Dead Cell Stain Kit (405 nm, Invitrogen) followed by 30 minutes staining in FACS buffer (PBS with 0.5% BSA and 2 mM EDTA) at room temperature with the following antibodies: CD3 (UCHT1, BD Biosciences), CD56 (N901, Beckman Coulter), CD4 (VIT4, Miltenyi Biotec), CD8 (RPA-T8, Biolegend), TCR-γδ (B1, Biolegend), TCR-VS1 (REA173, Miltenyi Biotec), KIR2DL1/S1 (EB6B, Beckman Coulter), KIR2DL2/L3/S2 (CH-L, BD Biosciences), KIR3DL1 (DX9, BD Biosciences), NKG2C (REA205, Miltenyi Biotec), NKG2A (Z199, Beckman Coulter), PD-1 (EH12.2H7, Biolegend). FcεRIγ (rabbit polyclonal, Millipore Sigma) was detected by intracellular staining using the FIX & PERM™ Cell Permeabilization Kit (Thermo Fisher).

For BCL11B staining, 1×10⁶ cells per well previously stained for extracellular markers as indicated above, were fixed and permeabilized using BD Phosflow kit (BD Bioscience). After two washes, intracellular staining was performed using BCL11B antibody (Abcam) or a Rat IgG2a Isotype control (Abcam) in FACS buffer.

TCR repertoire studies were performed using a IOTest® Beta Mark TCR Vβ Repertoire Kit (Beckman Coulter). For T cell differentiation analysis, 2×10⁵ PBMC were stained with the following antibodies: CD3 (UCHT1, BD Biosciences), CD8 (RPA-T8, Biolegend), CD4 (VIT4, Miltenyi Biotec), CD45RO (UCHL1, Biolegend), CD45RA (HI100, Biolegend), CD27 (M-T271, Biolegend), CD28 (CD28.2, Biolegend), CD197 (G043H7, Biolegend), CD95 (DX2, Biolegend), CD127 (A019D5, Biolegend), CD57 (HNK-1, Biolegend), CD62L (DREG-56, Biolegend). The following tetrameric peptide/HLA class I tetramer complexes from MBL® International Corporation were used in this study: CMV pp65 NLVPMVATV/HLA A*0201 (NLVPMVATV is SEQ ID NO. 4) and CMV pp65 TPRVTGGGAM/HLA B*0702 (TPRVTGGGAM is SEQ ID NO. 5).

For HLA-E surface detection on tumor cell lines, clone 3D12 (Biolegend) was used.

All FACS analyses were performed on an LSR Fortessa (BD Biosciences) and analyzed using FlowJo software (10.6.2).

### Cell Lines and Culture

Human acute myelogenous leukemia (AML) cell lines KG1, HL-60, THP-1, MOLM-13, U-937, SET-2 and the erythroleukemia cell line K562 expressing HLA-E with the HLA-G*01 leader sequence peptide (VMAPRTLFL (SEQ ID NO. 1)) were cultured in RPMI supplemented with 10% FCS and 1% Penicillin-Streptomycin. All the AML cell lines were stably transduced with a GFP-firefly luciferase fusion protein as described elsewhere(52).

721.221 and 721.221 transfected with cDNA encoding HLA-Cw3 or HLA-Cw4 were cultured in RPMI supplemented with 10% FCS and 1% Penicillin-Streptomycin.

The HLA-E*01:01 gene with the signal peptide exchanged for that of HLA-G*01 (MVVMAPRTLFLLLSGALTLTETWA, SEQ ID NO. 6) was codon-optimized for human translation and synthesized in pUC57 (Genscript). Site-directed mutagenesis to convert HLA-E*01:01 for HLA-E*01:03 (R128G) was performed using the Q5 Site-Directed Mutagenesis Kit (New England Biolabs) with the forward primer 5′-GGGACCAGACGGGAGATTCCTG-3′ (SEQ ID NO. 7) and reverse primer 5′-AGCTCGCATCCGTGCATC-3′ (SEQ ID NO. 8) (IDT). The HLA-E*01:03-HLA-G*01 gene was then cloned in the 3rd generation lentivector pERRL by restriction digest cloning with XbaI and SalI (NEB); pERRL was derived from pRRLSIN.cPPT.PGK-GFP.WPRE (Addgene #12252), exchanging the human PGK promoter for the human EF1α promoter and inserting an XbaI restriction site in the 5′ MCS. Lentivirus was produced as previously described(53); briefly, 293T cells (ATCC) were transiently transfected with 15 μg pERRL, 18 μg pRSV-Rev (Addgene #12253), 18 μg pMDLg/pRRE (Addgene #12251), and 7 μg pCI-VSVG (Addgene #1733) using Lipofectamine 2000 (Invitrogen), supernatants were collected at 24 and 48 hr post-transfection and concentrated by centrifugation at ≥30,000 g for ≥6 hr. Viral pellets were combined and resuspended in RPMI complete media and used to transduce parental K562 cells or K562 Clone 9 mbIL-21 cells. Transduced K562s were sorted by FACS for uniform high expression of HLA-E, while a high-expressing clone of transduced K562 Clone 9 mbIL-21 was isolated by limiting dilution and grown out.

Human primary fibroblasts cultures were obtained by outgrowth of cells from explanted skin biopsies(54). Briefly, after dissection, the dermis was minced into 1 mm³ pieces and 8 fragments were plated on the bottom of a 75 cm² culture dish. Explants were let air dry for 15 minutes and then 8 mL of DMEM supplemented with 20% FCS was added. After 2 weeks cells were dissociated with trypsin-EDTA. Human fibroblasts were infected with the HCMV TB40/E strain derivative TB40-BAC_{KL7}-SE-EGFP as previously described(27). The percentage of infected cells was assessed by flow cytometry (EGFP) prior to functional experiments.

For T cell proliferation analysis, 2×10⁵ CellTrace™ Violet (Invitrogen) dye-labelled PBMC were cultured in 50 U/mL IL-2 and stimulated with CD3/CD2/CD28 beads (Miltenyi Biotec) at a bead-to-cell ratio of 1:2 or irradiated K562 HLA-E:VMAPRTLFL (SEQ ID NO. 1) at a ratio of 1:1 for 7 days, replenishing the IL-2 and irradiated K562 HLA-E:VMAPRTLFL (SEQ ID NO. 1) four days post-stimulation. Responder frequency, proliferation index, and division index were calculated as previously described(55).

For the experiments were CAR T cells were used, NKG2C⁺ and NKG2C⁻CD8⁺ T cells were first FACS-sorted and stimulated with CD3/CD2/CD28 beads (Miltenyi Biotec) at a bead-to-cell ratio of 1:2 for 48 h in the presence of 100 U/ml IL2. After 48 h, beads were magnetically removed and cells were transduced as previously described(56). Prior to functional experiments cells were expanded for 1 week with K562 mbIL21 HLA-E:VMAPRTLFL (SEQ ID NO. 1) at a ratio of 1:1, in the presence of 100 U/ml IL2.

### Functional Assays

CD107a mobilization and IFN-γ production were used to determine CD8 T cell activation. Frozen PBMC samples were thawed and rested overnight in RPMI complete media with 200 U/mL IL-2 in a 37° C. incubator with 5% CO₂. PBMC (2×10⁵ cells per well) were incubated in 96-V bottom plates with target cells at a 5:1 ratio in the presence of anti-CD107a antibody (BD Biosciences). After 2 hours of co-culture, 55.5 μg/mL of Brefeldin A (MP Biomedicals) was added to the cells. After additional 4 hours of co-culture, cells were washed, fixed/permeabilized and stained with anti-IFNγ antibody (BD Biosciences). For plate-bound Ab stimulation assays, 96-well flat-bottom plates were pre-coated overnight at 4° C. with the following antibodies (10 g/mL): anti-NKG2C (134522, R&D systems), anti-NKG2D (1D11, Biolegend), anti-DNAM1 (DX11, BD Biosciences) or MICA-Fc (1300-MA, R&D systems). 2×10⁵ cells were added to the wells in complete medium containing anti-CD107a antibody. After 2 h of incubation, Brefeldin A was added and incubation followed for 4 additional hours at 37° C. Afterwards, cells were washed, fixed/permeabilized and stained with anti-IFN-γ antibody. In blocking experiments, the following antibodies (10 g/mL) were added at the beginning of the co-culture: anti-CD94 (HP-3B1, Santa Cruz Biotechnology), anti-NKG2D (1D11, Biolegend) or anti-DNAM1 (DX11, BD Biosciences) and the procedure continued as described above. The anti-CD94 antibody was first digested to purify F(ab′)2 fragments using the Pierce™ F(ab′)2 Preparation Kit (Thermo Fisher Scientific). For HCMV antigen-specific stimulation, T cells were stimulated with 1 μg/mL of the indicated ORFs (JPT), containing 15 amino acid peptides spanning the complete amino acid sequence of the indicated protein antigen. Following O/N peptide stimulation cells were stained with anti-IFN-γ antibody.

### RNA-Seq Processing and Analysis

Paired-end reads were trimmed for adaptors and removed of low quality reads using Trimmomatic (v0.38)(57). Transcript quantification was based on the hg38 UCSC Known Gene models and performed using the quasi-mapping-based mode of Salmon (v.0.10.2)(58), correcting for potential GC bias. Counts were summarized to the gene level using tximport (v1.10.1)(59). For those samples that were sequenced across two runs, summarized reads determined by tximport were summed, and the means of average transcript length offsets calculated for each run was used for downstream differential analyses executed by DESeq2 (v1.22.2)(60). Genes were considered differentially expressed (DE) if they showed a false discovery rate (FDR)-adjusted P-value <0.05. Gene set analysis was performed with GOseq (v.1.34.1)(61), using either DE genes higher in NKG2C⁺CD8⁺ T cells or DE genes higher in NKG2C⁻ NKG2A⁻ (DN) CD8⁺ T cells, and those showing an absolute log 2 fold change >1. Gene sets were retrieved from the MSigDB database (v.3.0)(62, 63). FDR-corrected P-values were calculated from P-values calculated by GOseq, and only gene ontologies passing a threshold of P<0.05 were considered. Up to the top 10 gene sets ranked on p-value are shown. For transcription factor assignment, a list of curated human genes associated with transcription factors were obtained from the Animal TFDB (v3.0)(64) and gene IDs were matched to DE gene IDs. To determine potential BCL11B-dependent genes, DE genes that were defined to be either repressed (upregulated in BCL11B-deficiency) or activated (downregulated in BCL11B-deficiency) were retrieved from a previously published study on mouse thymocytes(22). These gene names were converted into human orthologs using the NCBI gene IDs from the HCOP database(65), in addition to matching gene symbols between orthologs. Hypergeometric tests were calculated on the overlap between BCL11B-dependent genes and DE genes (NKG2C⁺ versus DN) using all the sufficiently expressed genes (18,943) that remained after independent filtering performed by DESeq2 as the total population size. All heatmaps were generated using ComplexHeatmap (v1.99.7)(66).

### Gene Targeting

For BCL11B locus targeting, NKG2A⁺NKG2C⁻KIRs⁻CD8⁺ T cells were FACS-sorted and left in RPMI complete media supplemented with 200 U/ml IL-2 O/N at a density of 10⁶ cells per ml. The next day, cells were transfected by electrotransfer of Cas9 protein and gRNA using a Nucleofector II Arnaxa system (Lonza). 5×10⁶ cells were mixed with 10 μg TrueCut Cas9 protein (Thermo Fisher Scientific) for each sgRNA and either 2 μl (200 pmol) of a control (TrueGuide synthetic sgRNA, Thermo Fisher Scientific) or 2 μl (200 pmol) each of three different BCL11B sgRNA guides (TrueGuide synthetic sgRNA, Thermo Fisher Scientific) into a 1 ml cuvette. The target DNA sequences of the BCL11B sgRNA guides were: 5′-CGCCATCCTCGAAGAAGACG-3′ SEQ ID NO. 9), 5′-GTTCATTTGACACTGGCCAC-3′ (SEQ ID NO. 10), 5′ACTTGGATCCCGATCTCCAC-3′ (SEQ ID NO. 11). Following electroporation, cells were diluted in RPMI complete medium supplemented with 100 U/ml IL-2 and incubated at 37° C., 5% CO₂. 48 h after electrotransfer cells were stimulated with CD3/CD2/CD28 beads (Miltenyi Biotec) at a bead-to-cell ratio of 1:2 and, after two weeks, with irradiated K562 mbIL21 HLA-E:VMAPRTLFL (SEQ ID NO. 1) at a ratio of 1:1 replenishing the IL-2 every 3 to 4 days and irradiated K562 every 7 days. For TRAC locus targeting, total T lymphocytes were purified using the EasySep™ Human T Cell Enrichment Kit (STEMCELL Technologies) and activated with CD3/CD2/CD28 beads (Miltenyi Biotec) at a bead-to-cell ratio of 1:2. 48 hours after initiating T cell activation, beads were magnetically removed and 2×10⁶ cells were transfected by electrotransfer of Cas9 protein and gRNA using a 4D Nucleofector X Unit system (Lonza) as described elsewhere(67).

### Short-Term Quantitative Cytotoxicity Assay

The short-term cytotoxicity of NKG2C⁻ or NKG2C⁺CD8 T cells was determined by a standard luciferase-based killing assay. 5×10³ target tumor cells expressing firefly luciferase were co-cultured with FACS sorted NKG2C⁻ or NKG2C⁺CD8 T cells at different effector-to-target ratios in triplicates in white-walled 96-well plates (Corning) in a total volume of 200 ul of cell media. Target cells alone were plated at the same cell density to determine the maximal luciferase expression as a reference (“max signal”), and 16 h later, 75 ng of D-Luciferin (Gold Biotechnology) dissolved in 50 ul of PBS was added to each well. Emitted luminescence of each sample (“sample signal”) was detected in a Spark plate reader (Tecan) and quantified using the SparkControl software (Tecan). Percent lysis was determined as (1−(“sample signal”/“max signal”))×100.

### Statistical Analysis

Paired t-test was applied for the comparison between groups with observations from the same donor cells. Unpaired t-test was used to compare independent groups. ANOVA with multiple comparisons was used to analyze groups of more than two. All tests are indicated in each individual figure legend. *P≤0.05, **P≤0.01, ***P≤0.001 and ****P≤0.0001 were used as significant p values. The analysis was performed using Prism 8 software (GraphPad).

TABLE 3
Sequences

| SEQ ID NO. | Sequence | Type |
|---|---|---|
| SEQ ID NO. 1 | VMAPRTLFL | Amino Acid |
| | | (Peptide) |
| SEQ ID NO. 2 | VMAPRTLIL | Amino Acid |
| | | (Peptide) |
| SEQ ID NO. 3 | VMAPRTLVL | Amino Acid |
| | | (Peptide) |
| SEQ ID NO. 4 | NLVPMVATV | Amino Acid |
| | | (Peptide) |
| SEQ ID NO. 5 | TPRVTGGGA | Amino Acid |
| | M | (Peptide) |
| SEQ ID NO. 6 | MVVMAPRTL | Amino Acid |
| | FLLLSGALT | (Peptide) |
| | LTETWA | |
| SEQ ID NO. 7 | 5′GGGACCA | Nucleotide |
| | GACGGGAGA | (Forward primer) |
| | TTCCTG-3′ | |
| SEQ ID NO. 8 | 5′-AGCTCG | Nucleotide |
| | CATCCGTGC | (Reverse primer) |
| | ATC-3′ | |
| SEQ ID NO. 9 | 5′-CGCCAT | Nucleotide |
| | CCTCGAAGA | (sgRNA guide) |
| | AGACG-3′ | |
| SEQ ID NO. 10 | 5′-GTTCAT | Nucleotide |
| | TTGACACTG | (sgRNA guide) |
| | GCCAC-3′ | |
| SEQ ID NO. 11 | 5′-ACTTGG | Nucleotide |
| | ATCCCGATC | (sgRNA guide) |
| | TCCAC-3′ | |

### REFERENCES

1. G. Picarda, C. A. Benedict, Cytomegalovirus: Shape-Shifting the Immune System. J Immunol 200, 3881-3889 (2018).
2. J. Dowd, A. Aiello, D. Alley, Socioeconomic disparities in the seroprevalence of cytomegalovirus infection in the US population: NHANES III. Epidemiol Infect 137, 58-65 (2009).
3. C. Falk, M. Mach, D. Schendel, E. Weiss, I. Hilgert, G. Hahn, NK cell activity during human cytomegalovirus infection is dominated by US2-11-mediated HLA class I down-regulation. J Immunol 169, 3257-3266 (2002).
4. J. Sun, J. Beilke, N. Bezman, L. Lanier, Homeostatic proliferation generates long-lived natural killer cells that respond against viral infection. J Exp Med 208, 357-368 (2011).
5. V. Prod'homme, P. Tomasec, C. Cunningham, M. K. Lemberg, R. J. Stanton, B. P. McSharry, E. C. Wang, S. Cuff, B. Martoglio, A. J. Davison, V. M. Braud, G. W. Wilkinson, Human cytomegalovirus UL40 signal peptide regulates cell surface expression of the NK cell ligands HLA-E and gpUL18. J Immunol 188, 2794-2804 (2012).
6. Q. Hammer, T. Rückert, E. Borst, J. Dunst, A. Haubner, P. Durek, F. Heinrich, G. Gasparoni, M. Babic, A. Tomic, G. Pietra, M. Nienen, I. Blau, J. Hofmann, I. Na, I. Prinz, C. Koenecke, P. Hemmati, N. Babel, R. Arnold, J. Walter, K. Thurley, M. Mashreghi, M. Messerle, C. Romagnani, Peptide-specific recognition of human cytomegalovirus strains controls adaptive natural killer cells. Nat Immunol 19, 453-463 (2018).
7. A. Rolle, J. Pollmann, E. Ewen, V. Le, A. Halenius, H. Hengel, A. Cerwenka, IL-12-producing monocytes and HLA-E control HCMV-driven NKG2C+ NK cell expansion. J Clin Invest 124, 5305-5316 (2014).
8. M. Guma, A. Angulo, C. Vilches, N. Gomez-Lozano, N. Malats, M. Lopez-Botet, Imprint of human cytomegalovirus infection on the NK cell receptor repertoire. Blood 104, 3664-3671 (2004).
9. H. Schlums, F. Cichocki, B. Tesi, J. Theorell, V. Beziat, T. Holmes, H. Han, S. Chiang, B. Foley, K. Mattsson, S. Larsson, M. Schaffer, K.-J. Malmberg, H.-G. Ljunggren, J. Miller, Y. Bryceson, Cytomegalovirus infection drives adaptive epigenetic diversification of NK cells with altered signaling and effector function. Immunity 42, 443-456 (2015).
10. J. Lee, T. Zhang, I. Hwang, A. Kim, L. Nitschke, M. Kim, J. Scott, Y. Kamimura, L. Lanier, S. Kim, Epigenetic modification and antibody-dependent expansion of memory-like NK cells in human cytomegalovirus-infected individuals. Immunity 42, 431-442 (2014).
11. P. Klenerman, A. Oxenius, T cell responses to cytomegalovirus. Nat Rev Immunol 16, 367-377 (2016).
12. A. Sylwester, B. Mitchell, J. Edgar, C. Taormina, C. Pelte, F. Ruchti, P. Sleath, K. Grabstein, N. Hosken, F. Kern, J. Nelson, L. Picker, Broadly targeted human cytomegalovirus-specific CD4+ and CD8+ T cells dominate the memory compartments of exposed subjects. J Exp Med 202, 673-685 (2005).
13. C. McMahon, A. Zajac, A. Jamieson, L. Corral, G. Hammer, R. Ahmed, D. Raulet, Viral and bacterial infections induce expression of multiple NK cell receptors in responding CD8(+) T cells. J Immunol 169, 1444-1452 (2002).
14. M. Mingari, F. Schiavetti, M. Ponte, C. Vitale, E. Maggi, S. Romagnani, J. Demarest, G. Pantaleo, A. Fauci, L. Moretta, Human CD8+ T lymphocyte subsets that express HLA class I-specific inhibitory receptors represent oligoclonally or monoclonally expanded cell populations. Proceedings of the National Academy of Sciences 93, 12433-12438 (1996).
15. N. Bjorkstrom, V. Béziat, F. Cichocki, L. Liu, J. Levine, S. Larsson, R. Koup, S.

Anderson, H. Ljunggren, K. Malmberg, CD8 T cells express randomly selected KIRs with distinct specificities compared with NK cells. Blood 120, 3455-3465 (2012).
16. M. Guma, L. Busch, L. Salazar-Fontana, B. Bellosillo, C. Morte, P. Garcia, M. Lopez-Botet, The CD94/NKG2C killer lectin-like receptor constitutes an alternative activation pathway for a subset of CD8+ T cells. Eur J Immunol 35, 2071-2080 (2005).
17. S. Balin, M. Pellegrini, E. Klechevsky, S. Won, D. Weiss, A. Choi, J. Hakimian, J. Lu, M. Ochoa, B. Bloom, L. Lanier, S. Stenger, R. Modlin, Human antimicrobial cytotoxic T lymphocytes, defined by NK receptors and antimicrobial proteins, kill intracellular bacteria. Sci Immunol 3, eaat7668, DOI: 7610.1126/sciimmunol.aat7668 (2018).
18. M. R. Wills, A. J. Carmichael, K. Mynard, X. Jin, M. P. Weekes, B. Plachter, J. G. Sissons, The human cytotoxic T-lymphocyte (CTL) response to cytomegalovirus is dominated by structural protein pp65: frequency, specificity, and T-cell receptor usage of pp65-specific CTL. J Virol 70, 7569-7579 (1996).
19. E. McLaughlin-Taylor, H. Pande, S. J. Forman, B. Tanamachi, C. R. Li, J. A. Zaia, P. D. Greenberg, S. R. Riddell, Identification of the major late human cytomegalovirus matrix protein pp65 as a target antigen for CD8+ virus-specific cytotoxic T lymphocytes. J Med Virol 43, 103-110 (1994).
20. F. Borrego, M. Masilamani, A. I. Marusina, X. Tang, J. E. Coligan, The CD94/NKG2 family of receptors: from molecules and cells to clinical relevance. Immunol Res 35, 263-278 (2006).
21. J. Duraiswamy, C. C. Ibegbu, D. Masopust, J. D. Miller, K. Araki, G. H. Doho, P. Tata, S. Gupta, M. J. Zilliox, H. I. Nakaya, B. Pulendran, W. N. Haining, G. J. Freeman, R. Ahmed, Phenotype, function, and gene expression profiles of programmed death-1(hi) CD8 T cells in healthy human adults. J Immunol 186, 4200-4212 (2011).
22. H. Hosokawa, M. Romero-Wolf, M. Yui, J. Ungerbäck, M. Quiloan, M. Matsumoto, K. Nakayama, T. Tanaka, E. Rothenberg, BCL11B sets pro-T cell fate by site-specific cofactor recruitment and by repressing Id2 and Zbtb16. Nature 19, 1427-1440 (2018).
23. L. Li, M. Leid, E. V. Rothenberg, An early T cell lineage commitment checkpoint dependent on the transcription factor BCL11B. Science 329, 89-93 (2010).
24. V. Pulko, J. S. Davies, C. Martinez, M. C. Lanteri, M. P. Busch, M. S. Diamond, K. Knox, E. C. Bush, P. A. Sims, S. Sinari, D. Billheimer, E. K. Haddad, K. O. Murray, A. M. Wertheimer, J. Nikolich-Zugich, Human memory T cells with a naive phenotype accumulate with aging and respond to persistent viruses. Nat Immunol 17, 966-975 (2016).
25. R. Zeng, R. Spolski, S. E. Finkelstein, S. Oh, P. E. Kovanen, C. S. Hinrichs, C. A. Pise-Masison, M. F. Radonovich, J. N. Brady, N. P. Restifo, J. A. Berzofsky, W. J. Leonard, Synergy of IL-21 and IL-15 in regulating CD8+ T cell expansion and function. J Exp Med 201, 139-148 (2005).
26. E. Mahe, T. Pugh, S. Kamel-Reid, T cell clonality assessment: past, present and future. J Clin Pathol 71, 195-200 (2018).
27. K. L. Sampaio, A. Weyell, N. Subramanian, Z. Wu, C. Sinzger, A TB40/E-derived human cytomegalovirus genome with an intact US-gene region and a self-excisable BAC cassette for immunological research. Biotechniques 63, 205-214 (2017).
28. N. Lee, D. R. Goodlett, A. Ishitani, H. Marquardt, D. E. Geraghty, HLA-E surface expression depends on binding of TAP-dependent peptides derived from certain HLA class I signal sequences. J Immunol 160, 4951-4960 (1998).
29. M. P. Correia, A. Stojanovic, K. Bauer, D. Juraeva, L. O. Tykocinski, H. M. Lorenz, B. Brors, A. Cerwenka, Distinct human circulating NKp30(+)FcepsilonRIgamma(+)CD8(+) T cell population exhibiting high natural killer-like antitumor potential. Proc Natl Acad Sci USA 115, E5980-E5989 (2018).
30. L. Arlettaz, J. Villard, C. de Rham, S. Degermann, B. Chapuis, B. Huard, E. Roosnek, Activating CD94:NKG2C and inhibitory CD94:NKG2A receptors are expressed by distinct subsets of committed CD8+ TCR alphabeta lymphocytes. Eur J Immunol 34, 3456-3464 (2004).
31. B. Meresse, S. A. Curran, C. Ciszewski, G. Orbelyan, M. Setty, G. Bhagat, L. Lee, M. Tretiakova, C. Semrad, E. Kistner, R. J. Winchester, V. Braud, L. L. Lanier, D. E. Geraghty, P. H. Green, S. Guandalini, B. Jabri, Reprogramming of CTLs into natural killer-like cells in celiac disease. J Exp Med 203, 1343-1355 (2006).
32. A. R. Manser, N. Scherenschlich, C. Thons, H. Hengel, J. Timm, M. Uhrberg, KIR Polymorphism Modulates the Size of the Adaptive NK Cell Pool in Human Cytomegalovirus-Infected Individuals. J Immunol 203, 2301-2309 (2019).
33. S. E. Jackson, G. X. Sedikides, G. Okecha, M. R. Wills, Generation, maintenance and tissue distribution of T cell responses to human cytomegalovirus in lytic and latent infection. Med Microbiol Immunol 208, 375-389 (2019).
34. V. M. Braud, D. S. Allan, A. O'Callaghan, K. Soderstrom, A. D'Andrea, G. S. Ogg, S. Lazetic, N. T. Young, J. I. Bell, J. H. Phillips, HLA-E binds to natural killer cell receptors CD94/NKG2A, B and C. Nature 391, 795-799 (1998).
35. N. Anfossi, J. M. Doisne, M. A. Peyrat, S. Ugolini, O. Bonnaud, D. Bossy, V. Pitard, P. Merville, J. F. Moreau, J. F. Delfraissy, J. Dechanet-Merville, M. Bonneville, A. Venet, E. Vivier, Coordinated expression of Ig-like inhibitory MHC class I receptors and acquisition of cytotoxic function in human CD8+ T cells. J Immunol 173, 7223-7229 (2004).
36. M. C. Mingari, C. Vitale, A. Cambiaggi, F. Schiavetti, G. Melioli, S. Ferrini, A. Poggi, Cytolytic T lymphocytes displaying natural killer (NK)-like activity: expression of NK-related functional receptors for HLA class I molecules (p58 and CD94) and inhibitory effect on the TCR-mediated target cell lysis or lymphokine production. Int Immunol 7, 697-703 (1995).
37. J. P. Bottcher, M. Beyer, F. Meissner, Z. Abdullah, J. Sander, B. Hochst, S. Eickhoff, J. C. Rieckmann, C. Russo, T. Bauer, T. Flecken, D. Giesen, D. Engel, S. Jung, D. H. Busch, U. Protzer, R. Thimme, M. Mann, C. Kurts, J. L. Schultze, W. Kastenmuller, P. A. Knolle, Functional classification of memory CD8(+) T cells by CX3CR1 expression. Nat Commun 6, 8306 (2015).
38. S. Simon, V. Vignard, L. Florenceau, B. Dreno, A. Khammari, F. Lang, N. Labarriere, PD-1 expression conditions T cell avidity within an antigen-specific repertoire. Oncoimmunology 5, e1104448 (2016).
39. A. Gros, M. R. Parkhurst, E. Tran, A. Pasetto, P. F. Robbins, S. Ilyas, T. D. Prickett, J. J. Gartner, J. S. Crystal, I. M. Roberts, K. Trebska-McGowan, J. R. Wunderlich, J. C. Yang, S. A. Rosenberg, Prospective identification of neoantigen-specific lymphocytes in the peripheral blood of melanoma patients. Nat Med 22, 433-438 (2016).
40. R. J. Martinez, B. D. Evavold, Lower Affinity T Cells are Critical Components and Active Participants of the Immune Response. Front Immunol 6, 468 (2015).
41. Y. Wakabayashi, H. Watanabe, J. Inoue, N. Takeda, J. Sakata, Y. Mishima, J. Hitomi, T. Yamamoto, M. Utsuyama, O. Niwa, S. Aizawa, R. Kominami, BCL11B is required for differentiation and survival of alphabeta T lymphocytes. Nat Immunol 4, 533-539 (2003).
42. L. Li, M. Leid, E. Rothenberg, An early T cell lineage commitment checkpoint dependent on the transcription factor BCL11B. Science 329, 89-93 (2010).
43. B. Huard, L. Karlsson, F. Triebel, KIR down-regulation on NK cells is associated with down-regulation of activating receptors and NK cell inactivation. Eur J Immunol 31, 1728-1735 (2001).
44. F. Borrego, J. Kabat, T. B. Sanni, J. E. Coligan, NK cell CD94/NKG2A inhibitory receptors are internalized and recycle independently of inhibitory signaling processes. J Immunol 169, 6102-6111 (2002).
45. S. J. Judge, C. Dunai, E. G. Aguilar, S. C. Vick, I. R. Sturgill, L. T. Khuat, K. M. Stoffel, J. Van Dyke, D. L. Longo, M. A. Darrow, S. K. Anderson, B. R. Blazar, A. M. Monjazeb, J. S. Serody, R. J. Canter, W. J. Murphy, Minimal PD-1 expression in mouse and human NK cells under diverse conditions. J Clin Invest 130, 3051-3068 (2020).
46. P. Tomasec, V. Braud, C. Rickards, M. Powell, B. McSharry, S. Gadola, V. Cerundolo, L. Borysiewicz, A. McMichael, G. Wilkinson, Surface expression of HLA-E, an inhibitor of natural killer cells, enhanced by human cytomegalovirus gpUL40. Science 287, 1031 (2000).
47. M. Llano, N. Lee, F. Navarro, P. Garcia, J. P. Albar, D. E. Geraghty, M. Lopez-Botet, HLA-E-bound peptides influence recognition by inhibitory and triggering CD94/NKG2 receptors: preferential response to an HLA-G-derived nonamer. Eur J Immunol 28, 2854-2863 (1998).
48. M. Vales-Gomez, H. T. Reyburn, R. A. Erskine, M. Lopez-Botet, J. L. Strominger, Kinetics and peptide dependency of the binding of the inhibitory NK receptor CD94/NKG2-A and the activating receptor CD94/NKG2-C to HLA-E. EMBO J 18, 4250-4260 (1999).
49. P. Andre, C. Denis, C. Soulas, C. Bourbon-Caillet, J. Lopez, T. Arnoux, M. Blery, C. Bonnafous, L. Gauthier, A. Morel, B. Rossi, R. Remark, V. Breso, E. Bonnet, G. Habif, S. Guia, A. I. Lalanne, C. Hoffmann, O. Lantz, J. Fayette, A. Boyer-Chammard, R. Zerbib, P. Dodion, H. Ghadially, M. Jure-Kunkel, Y. Morel, R. Herbst, E. Narni-Mancinelli, R. B. Cohen, E. Vivier, Anti-NKG2A mAb Is a Checkpoint Inhibitor that Promotes Anti-tumor Immunity by Unleashing Both T and NK Cells. Cell 175, 1731-1743 e1713 (2018).
50. M. Busch, C. Herzmann, S. Kallert, A. Zimmermann, C. Hofer, D. Mayer, S. F. Zenk, R. Muche, C. Lange, B. R. Bloom, R. L. Modlin, S. Stenger, T. B. Network, Lipoarabinomannan-Responsive Polycytotoxic T Cells Are Associated with Protection in Human Tuberculosis. Am J Respir Crit Care Med 194, 345-355 (2016).
51. K. Hikami, N. Tsuchiya, T. Yabe, K. Tokunaga, Variations of human killer cell lectin-like receptors: common occurrence of NKG2-C deletion in the general population. Genes Immun 4, 160-167 (2003).
52. H. J. Pegram, T. J. Purdon, D. G. van Leeuwen, K. J. Curran, S. A. Giralt, J. N. Barker, R. J. Brentjens, IL-12-secreting CD19-targeted cord blood-derived T cells for the immunotherapy of B-cell acute lymphoblastic leukemia. Leukemia 29, 415-422 (2015).
53. R. V. Parry, C. A. Rumbley, L. H. Vandenberghe, C. H. June, J. L. Riley, CD28 and inducible costimulatory protein Src homology 2 binding domains show distinct regulation of phosphatidylinositol 3-kinase, Bcl-xL, and IL-2 expression in primary human CD4 T lymphocytes. J Immunol 171, 166-174 (2003).
54. L. Rittie, G. J. Fisher, Isolation and culture of skin fibroblasts. Methods Mol Med 117, 83-98 (2005).
55. A. B. Lyons, Analysing cell division in vivo and in vitro using flow cytometric measurement of CFSE dye dilution. J Immunol Methods 243, 147-154 (2000).
56. E. L. Smith, K. Harrington, M. Staehr, R. Masakayan, J. Jones, T. J. Long, K. Y. Ng, M. Ghoddusi, T. J. Purdon, X. Wang, T. Do, M. T. Pham, J. M. Brown, C. F. De Larrea, E. Olson, E. Peguero, P. Wang, H. Liu, Y. Xu, S. C. Garrett-Thomson, S. C. Almo, H. G. Wendel, I. Riviere, C. Liu, B. Sather, R. J. Brentjens, GPRC5D is a target for the immunotherapy of multiple myeloma with rationally designed CAR T cells. Sci Transl Med 11, (2019).
57. A. M. Bolger, M. Lohse, B. Usadel, Trimmomatic: a flexible trimmer for Illumina sequence data. Bioinformatics 30, 2114-2120 (2014).
58. R. Patro, G. Duggal, M. I. Love, R. A. Irizarry, C. Kingsford, Salmon provides fast and bias-aware quantification of transcript expression. Nat Methods 14, 417-419 (2017).
59. C. Soneson, M. I. Love, M. D. Robinson, Differential analyses for RNA-seq: transcript-level estimates improve gene-level inferences. F1000Res 4, 1521 (2015).
60. M. I. Love, W. Huber, S. Anders, Moderated estimation of fold change and dispersion for RNA-seq data with DESeq2. Genome Biol 15, 550 (2014).
61. M. D. Young, M. J. Wakefield, G. K. Smyth, A. Oshlack, Gene ontology analysis for RNA-seq: accounting for selection bias. Genome Biol 11, R14 (2010).
62. A. Subramanian, P. Tamayo, V. K. Mootha, S. Mukherjee, B. L. Ebert, M. A. Gillette, A. Paulovich, S. L. Pomeroy, T. R. Golub, E. S. Lander, J. P. Mesirov, Gene set enrichment analysis: a knowledge-based approach for interpreting genome-wide expression profiles. Proc Natl Acad Sci USA 102, 15545-15550 (2005).
63. A. Liberzon, A. Subramanian, R. Pinchback, H. Thorvaldsdottir, P. Tamayo, J. P. Mesirov, Molecular signatures database (MSigDB) 3.0. Bioinformatics 27, 1739-1740 (2011).
64. H. Hu, Y. R. Miao, L. H. Jia, Q. Y. Yu, Q. Zhang, A. Y. Guo, AnimalTFDB 3.0: a comprehensive resource for annotation and prediction of animal transcription factors. Nucleic Acids Res 47, D33-D38 (2019).
65. B. Yates, B. Braschi, K. A. Gray, R. L. Seal, S. Tweedie, E. A. Bruford, Genenames.org: the HGNC and VGNC resources in 2017. Nucleic Acids Res 45, D619-D625 (2017).
66. Z. Gu, R. Eils, M. Schlesner, Complex heatmaps reveal patterns and correlations in multidimensional genomic data. Bioinformatics 32, 2847-2849 (2016).
67. J. Eyquem, J. Mansilla-Soto, T. Giavridis, S. J. van der Stegen, M. Hamieh, K. M. Cunanan, A. Odak, M. Gonen, M. Sadelain, Targeting a CAR to the TRAC locus with CRISPR/Cas9 enhances tumour rejection. Nature 543, 113-117 (2017).

We claim:

1. An engineered human T cell, wherein the T cell is NKG2C+ NKG2A−CD8+ and comprises a recombinant nucleic acid molecule encoding a chimeric antigen receptor (CAR) or a transgenic T cell receptor.

2. The engineered human T cell of claim 1, wherein the T cell is CD45RA+CD45RO−.

3. The engineered human T cell of claim 1, wherein the T cell is activated and is CD45RA−CD45RO+.

4. The engineered human T cell of claim 1, wherein the T cell is CCR7−

5. The engineered human T cell of claim 1, wherein the T cell is KIRs+.

6. The engineered human T cell of claim 1, wherein the T cell is BCL11B−.

7. The engineered human T cell of claim 1, wherein the T cell is CD45RA+ CD45RO− CCR7− KIRs+ BCL11B−.

8. The engineered human T cell of claim 1, wherein the T cell is activated and is CD45RA− CD45RO+ CCR7− KIRs+ BCL11B−.

9. The engineered human T cell of claim 1, wherein the T cell is positive for one or more markers selected from the group consisting of: KIR2DS4, SYK, ITGAX, KIR2DL3, S1PR5, PRF1, TBX21, NCR1, GZMB, GNLY, IL2RB, KIR2DL1, KIR2DL4, KIR3DL2, KLRC2, KLRC3, TYROBP, CD244, KLRC1, LAT2, KIR3DL1, PLCG2, FCGR3A, LYN, NCAM1, SLAMF7, JAZF1, SOX13, IKZF2, TBX21, ASCL2, L3MBTL4, HOPX, CD56, KIR, CD94, DAP12+ and TCRαβ.

10. The engineered human T cell of claim 1, wherein the T cell is negative for one or more markers selected from the group consisting of: TCF7, CD27, CD5, PDCD1, CD28, CCR7, CD6, IL7R, THEMIS, TESPA1, BCL11B, ZNF518B, SATB1, ZEB1, SREBF1, BACH2, FOXP1, YBX3, ARID5A, ZFP36L1, RERE, E2F3, KLF7, and PD1.

11. The engineered human T cell of claim 1, comprising a T cell receptor (TCR) comprising a TRBV-14 Vβ chain.

12. The engineered human T cell of claim 11, wherein the TCR is a transgenic TCR.

13. The engineered human T cell of claim 11, wherein the TCR is a native TCR.

14. The engineered human T cell of claim 1, comprising a T cell receptor (TCR) comprising a TRBV-28 Vβ chain.

15. The engineered human T cell of claim 14, wherein the TCR is a transgenic TCR.

16. The engineered human T cell of claim 14, wherein the TCR is a native TCR.

17. The engineered human T cell of any of the preceding claims, wherein expression of a native T cell receptor has been inhibited.

18. The engineered human T cell of any of the preceding claims, wherein the T cell has been genetically modified to knock out a native T cell receptor gene.

19. The engineered human T cell of claim 1, comprising a chimeric antigen receptor (CAR) that comprises an antigen-binding domain that binds to a tumor-associated antigen.

20. The engineered human T cell of claim 19, wherein the tumor-associated antigen is selected from the group consisting of CD19, CD20, CD22, CD30, CD33, BCMA, Igk and ROR1.

21. The engineered human T cell of claim 19, wherein the tumor-associated antigen is CD19.

22. The engineered human T cell of claim 21, wherein the CAR comprises the CDR1, CDR2 and CDR3 domains of the 1928z anti-CD19 CAR.

23. The engineered human T cell of claim 21, wherein the CAR is the 1928z anti-CD19 CAR.

24. A composition comprising a population of engineered human T cells according to any of the preceding claims.

25. The composition of claim 24, wherein at least 70% of the cells in the composition are NKG2C+ NKG2A−CD8+ T cells.

26. The composition of claim 24, wherein at least 80% of the cells in the composition are NKG2C+ NKG2A−CD8+ T cells.

27. The composition of claim 24, wherein at least 90% of the cells in the composition are NKG2C+ NKG2A−CD8+ T cells.

28. The composition of claim 24, wherein at least 95% of the cells in the composition are NKG2C+ NKG2A−CD8+ T cells.

29. The composition of claim 24, wherein at least 98% of the cells in the composition are NKG2C+ NKG2A−CD8+ T cells.

30. The composition of claim 24, wherein at least 99% of the cells in the composition are NKG2C+ NKG2A−CD8+ T cells.

31. The composition of claim 24, wherein 100% of the cells in the composition are NKG2C+ NKG2A−CD8+ T cells.

32. The composition of any of claims 24-31, for use in treating a tumor in a human subject in need thereof.

33. The composition of any of claims 24-31, for use in enhancing or stimulating an anti-tumor immune response in a human subject in need thereof.

34. The composition of any of claims 24-31, for use in treating a viral infection in a human subject in need thereof.

35. A method of killing a tumor cell, the method comprising contacting a tumor cell with an effective amount of a composition according to any of claims 24-31.

36. The method of claim 35, wherein the tumor cell is in vitro.

37. The method of claim 35, wherein the tumor cell is in vivo.

38. A method of killing a tumor cell, the method comprising contacting a tumor cell with an effective amount of a composition comprising engineered human T cells according to claim 1.

39. A method of killing a tumor cell, the method comprising contacting a tumor cell with an effective amount of a composition comprising engineered human T cells according to claim 21.

40. A method of killing a tumor cell, the method comprising contacting a tumor cell with an effective amount of a composition comprising engineered human T cells according to claim 22.

41. A method of killing a tumor cell, the method comprising contacting a tumor cell with an effective amount of a composition comprising engineered human T cells according to claim 23.

42. A method of treating a tumor in a human subject in need thereof, the method comprising administering to the subject an effective amount of a composition according to any of claims 24-31.

43. A method of treating a tumor, the method comprising contacting a tumor cell with an effective amount of a composition comprising engineered human T cells according to claim 1.

44. A method of treating a tumor, the method comprising contacting a tumor cell with an effective amount of a composition comprising engineered human T cells according to claim 21.

45. A method of treating a tumor, the method comprising contacting a tumor cell with an effective amount of a composition comprising engineered human T cells according to claim 22.

46. A method of treating a tumor, the method comprising contacting a tumor cell with an effective amount of a composition comprising engineered human T cells according to claim 23.

47. A method of enhancing or stimulating an anti-tumor immune response in subject in need thereof, the method comprising administering to the subject an effective amount of a composition according to any of claims 24-31.

48. A method of enhancing or stimulating an anti-tumor immune response in subject in need thereof, the method comprising contacting a tumor cell with an effective amount of a composition comprising engineered human T cells according to claim 1.

49. A method of enhancing or stimulating an anti-tumor immune response in subject in need thereof, the method comprising contacting a tumor cell with an effective amount of a composition comprising engineered human T cells according to claim 21.

50. A method of enhancing or stimulating an anti-tumor immune response in subject in need thereof, the method comprising contacting a tumor cell with an effective amount of a composition comprising engineered human T cells according to claim 22.

51. A method of enhancing or stimulating an anti-tumor immune response in subject in need thereof, the method comprising contacting a tumor cell with an effective amount of a composition comprising engineered human T cells according to claim 23.

52. A method of treating a viral infection in a human subject in need thereof, the method comprising administering to the subject an effective amount of a composition according to any of claims 24-31.

53. A method of treating a viral infection in a human subject in need thereof, the method comprising contacting a tumor cell with an effective amount of a composition comprising engineered human T cells according to claim 1.

54. A method of treating a viral infection in a human subject in need thereof, the method comprising contacting a tumor cell with an effective amount of a composition comprising engineered human T cells according to claim 21.

55. A method of treating a viral infection in a human subject in need thereof, the method comprising contacting a tumor cell with an effective amount of a composition comprising engineered human T cells according to claim 22.

56. A method of treating a viral infection in a human subject in need thereof, the method comprising contacting a tumor cell with an effective amount of a composition comprising engineered human T cells according to claim 23.

57. An adoptive cell therapy method for use in treating a human subject in need thereof, the method comprising administering NKG2C+ NKG2A− CD8+ T cells obtained from a donor subject to a recipient subject.

58. The adoptive cell therapy method of claim 57, comprising: (a) expanding and/or activating the NKG2C+ NKG2A−CD8+ T cells obtained from the donor subject ex vivo, and (b) administering the expanded and/or activated NKG2C+ NKG2A− CD8+ T cells to the recipient subject.

59. The adoptive cell therapy method of claim 57, comprising: (a) obtaining a population of T cells from a donor subject, (b) expanding and/or activating NKG2C+ NKG2A− CD8+ T cells present in the population of T cells ex vivo, and (c) administering the expanded and/or activated NKG2C+ NKG2A− CD8+ T cells to the recipient subject.

60. The adoptive cell therapy method of claim 57, comprising: (a) obtaining a mixed population of T cells from a donor subject, (b) isolating NKG2C+ NKG2A− CD8+ T cells from the mixed population of T cells, (c) expanding and/or activating the NKG2C+ NKG2A−CD8+ T cells ex vivo, and (d) administering the expanded and/or activated NKG2C+ NKG2A− CD8+ T cells to the recipient subject.

61. The adoptive cell therapy method of any of claims 57-60, wherein the T cells are CD45RA+ CD45RO−.

62. The adoptive cell therapy method of any of claims 57-60, wherein the T cells are activated and are CD45RA−CD45RO+.

63. The adoptive cell therapy method of any of claims 57-60, wherein the T cells are CCR7−.

64. The adoptive cell therapy method of any of claims 57-60, wherein the T cells are KIRs+.

65. The adoptive cell therapy method of any of claims 57-60, wherein the T cells are BCL11B−.

66. The adoptive cell therapy method of any of claims 57-60, wherein the T cells are CD45RA+ CD45RO− CCR7− KIRs+ BCL11B−.

67. The adoptive cell therapy method of any of claims 57-60, wherein the T cells are activated and is CD45RA− CD45RO+ CCR7− KIRs+ BCL11B−.

68. The adoptive cell therapy method of any of claims 57-60, wherein the T cells are positive for one or more markers selected from the group consisting of: KIR2DS4, SYK, ITGAX, KIR2DL3, S1PR5, PRF1, TBX21, NCR1, GZMB, GNLY, IL2RB, KIR2DL1, KIR2DL4, KIR3DL2, KLRC2, KLRC3, TYROBP, CD244, KLRC1, LAT2, KIR3DL1, PLCG2, FCGR3A, LYN, NCAM1, SLAMF7, JAZF1, SOX13, IKZF2, TBX21, ASCL2, L3MBTL4, HOPX, CD56, KIR, CD94, DAP12+ and TCRαβ.

69. The adoptive cell therapy method of any of claims 57-60, wherein the T cells are negative for one or more markers selected from the group consisting of: TCF7, CD27, CD5, PDCD1, CD28, CCR7, CD6, IL7R, THEMIS, TESPA1, BCL11B, ZNF518B, SATB1, ZEB1, SREBF1, BACH2, FOXP1, YBX3, ARID5A, ZFP36L1, RERE, E2F3, KLF7, and PD1.

70. The adoptive cell therapy method of any of claims 57-60, wherein the T cells comprise a transgenic T cell receptor (TCR).

71. The adoptive cell therapy method of any of claims 57-60, wherein the T cells comprise a T cell receptor (TCR) comprising a TRBV-14 Vβ chain.

72. The adoptive cell therapy method of claim 71, wherein the TCR is a transgenic TCR.

73. The adoptive cell therapy method of claim 71, wherein the TCR is a native, non-transgenic TCR.

74. The adoptive cell therapy method of any of claims 57-60, wherein the T cells comprise a T cell receptor (TCR) comprising a TRBV-28 Vβ chain.

75. The adoptive cell therapy method of claim 74, wherein the TCR is a transgenic TCR.

76. The adoptive cell therapy method of claim 74, wherein the TCR is a native, non-transgenic TCR.

77. The adoptive cell therapy method of any of claims 57-60, wherein expression of a native T cell receptor has been inhibited.

78. The adoptive cell therapy method of any of claims 57-60, wherein the T cell has been genetically modified to knock out a native T cell receptor gene.

79. The adoptive cell therapy method of any of claims 57-60, wherein the T cells comprise a chimeric antigen receptor.

80. The adoptive cell therapy method of any of claims 57-60, wherein the T cells comprise a chimeric antigen receptor that comprises an antigen-binding domain that binds to a tumor-associated antigen.

81. The adoptive cell therapy method of claim 80, wherein the tumor-associated antigen is selected from the group consisting of CD19, CD20, CD22, CD30, CD33, BCMA, Igk and ROR1.

82. The adoptive cell therapy method of claim 80, wherein the tumor-associated antigen is CD19.

83. The adoptive cell therapy method of claim 82, wherein the CAR comprises the CDR1, CDR2 and CDR3 domains of the 1928z anti-CD19 CAR.

84. The adoptive cell therapy method of claim 82, wherein the CAR is the 1928z anti-CD19 CAR.

85. A composition comprising a population of NKG2C+ NKG2A− CD8+ human T cells and one or more excipients.

86. The composition of claim 85, wherein the T cells are CD45RA+ CD45RO−.

87. The composition of claim 85, wherein the T cells are activated and are CD45RA− CD45RO+.

88. The composition of claim 85, wherein the T cells are CCR7−

89. The composition of claim 85, wherein the T cells are KIRs+.

90. The composition of claim 85, wherein the T cells are BCL11B−.

91. The composition of claim 85, wherein the T cells are CD45RA+ CD45RO− CCR7− KIRs+ BCL11B−.

92. The composition of claim 85, wherein the T cells are activated and are CD45RA− CD45RO+ CCR7− KIRs+ BCL11B−.

93. The composition of claim 85, wherein the T cells are positive for one or more markers selected from the group consisting of: KIR2DS4, SYK, ITGAX, KIR2DL3, S1PR5, PRF1, TBX21, NCR1, GZMB, GNLY, IL2RB, KIR2DL1, KIR2DL4, KIR3DL2, KLRC2, KLRC3, TYROBP, CD244, KLRC1, LAT2, KIR3DL1, PLCG2, FCGR3A, LYN, NCAM1, SLAMF7, JAZF1, SOX13, IKZF2, TBX21, ASCL2, L3MBTL4, HOPX, CD56, KIR, CD94, DAP12+ and TCRαβ.

94. The composition of claim 85, wherein the T cells are negative for one or more markers selected from the group consisting of: TCF7, CD27, CD5, PDCD1, CD28, CCR7, CD6, IL7R, THEMIS, TESPA1, BCL11B, ZNF518B, SATB1, ZEB1, SREBF1, BACH2, FOXP1, YBX3, ARID5A, ZFP36L1, RERE, E2F3, KLF7, and PD1.

95. The composition of any of claims 85-94, wherein the T cells comprise a transgenic T cell receptor (TCR).

96. The composition of any of claims 85-94, wherein the T cells comprise a TCR that comprises a TRBV-14 Vβ chain.

97. The composition of claim 96, wherein the TCR is a transgenic TCR.

98. The composition of claim 96, wherein the TCR is a native TCR.

99. The composition of any of claims 85-94, wherein the T cells comprise a TCR that comprises a TRBV-28 Vβ chain.

100. The composition of claim 99, wherein the TCR is a transgenic TCR.

101. The composition of claim 99, wherein the TCR is a native TCR.

102. The composition of any of claims 85-101, wherein expression of a native T cell receptor has been inhibited in the T cells.

103. The composition of any of claims 85-101, wherein the T cells have been genetically modified to knock out a native T cell receptor gene.

104. The composition of any of claims 85-103, wherein the T cells comprise a chimeric antigen receptor.

105. The composition of any of claims 85-103, wherein the T cells comprise a chimeric antigen receptor that comprises an antigen-binding domain that binds to a tumor-associated antigen.

106. The composition of claim 105, wherein the tumor-associated antigen is selected from the group consisting of CD19, CD20, CD22, CD30, CD33, BCMA, Igk and ROR1.

107. The composition of claim 105, wherein the tumor-associated antigen is CD19.

108. The composition of claim 107, wherein the CAR comprises the CDR1, CDR2 and CDR3 domains of the 1928z anti-CD19 CAR.

109. The composition of claim 107, wherein the CAR is the 1928z anti-CD19 CAR.

110. The composition of any of claims 85-109, wherein at least 70% of the cells in the composition are NKG2C+ NKG2A−CD8+ T cells.

111. The composition of any of claims 85-109, wherein at least 80% of the cells in the composition are NKG2C+ NKG2A−CD8+ T cells.

112. The composition of any of claims 85-109, wherein at least 90% of the cells in the composition are NKG2C+ NKG2A−CD8+ T cells.

113. The composition of any of claims 85-109, wherein at least 95% of the cells in the composition are NKG2C+ NKG2A−CD8+ T cells.

114. The composition of any of claims 85-109, wherein at least 99% of the cells in the composition are NKG2C+ NKG2A−CD8+ T cells.

115. The composition of any of claims 85-109, wherein 100% of the cells in the composition are NKG2C+ NKG2A−CD8+ T cells.

116. The composition of any of claims 85-115, wherein the composition is a therapeutic composition.

117. The composition of any of claims 85-116, for use in treating a tumor in a human subject in need thereof.

118. The composition of any of claims 85-116, for use in enhancing or stimulating an anti-tumor immune response in a human subject in need thereof.

119. The composition of any of claims 85-116, for use in treating a viral infection in a human subject in need thereof.

120. A method producing an NKG2C+CD8+ T cell from an NKG2C− CD8+ T cell, the method comprising knocking out the BCL11B gene in an NKG2C− CD8+ T cell, wherein following knock-out of the BCL11B gene, NKG2C is expressed thereby producing an NKG2C+ CD8+ T cell.

121. The method of claim 120, wherein the NKG2C− CD8+ T cell is in vivo.

122. The method of claim 120, wherein the NKG2C− CD8+ T cell is ex vivo.

123. A method producing an NKG2C+CD8+ T cell from an NKG2C−CD8+ T cell, the method comprising inhibiting expression of the BCL11B gene in an NKG2C− T cell, wherein following inhibition of expression of the BCL11B gene, NKG2C is expressed, thereby producing an NKG2C+ CD8+ T cell.

124. The method of claim 123, wherein the NKG2C− CD8+ T cell is in vivo.

125. The method of claim 123, wherein the NKG2C− CD8+ T cell is ex vivo.
